(19) United States
(12) Patent Application Publication
(10) Pub. No.: US 2025/0166736 A1
(43) Pub. Date: May 22, 2025

(54) RELATIONAL BIOMARKERS THAT DISTINGUISH DISEASES AND DISORDERS FROM CONTROLS AND USES THEREOF TO PREDICT PATHOPHYSIOLOGICAL OUTCOMES

(71) Applicant: Signature Diagnostics Inc., Pittsburgh, PA (US)

(72) Inventor: Paul COHEN, Pittsburgh, PA (US)

(21) Appl. No.: 18/514,319

(22) Filed: Nov. 20, 2023

(51) Int. Cl. G16B 40/00; G16B 5/00; G16H 50/20; G16H 50/30; G16H 50/70

(52) U.S. Cl. CPC G16B 40/00; G16B 5/00; G16H 50/20; G16H 50/30; G16H 50/70

(57) ABSTRACT

Methods for modeling system behavior and discovering relational biomarkers that distinguish a disease/disorder sample from a control sample. The methods generally include modeling a mathematical relationship between a pattern of a first biological material and the pattern(s) on one or more other biological samples for samples from each of a plurality of subjects having the disease/disorder to determine a case relational biomarker, and for samples from each of a plurality of subjects absent the disease/disorder to determine a control or noncase relational biomarker. These case noncase biomarkers, or discriminators, may be used to classify an unknown sample. Ensembles of discriminators may be generated by modeling the relationships among different combinations of biological materials. Moreover, ensembles of discriminators for various diseases or disorders, i.e., multiclass classifiers, may be generated by modeling the relationships among material from subjects who have or will develop additional diseases or disorders.

### TECHNICAL FIELD

The present disclosure relates to methods for classifying and predicting diseases and disorders using relational models of normal and abnormal system behavior. More specifically, the present disclosure relates to modeling mathematical functions that define relationships among patterns on two or more biological materials for the purpose of discriminating subjects who have, or will have, different health conditions. This disclosure covers case-noncase classifications that discriminate subjects who have or will develop a disease or disorder from those who do not have and will not develop the disease or disorder. The disclosure also covers classifications and predictions that identify subjects who do or do not have (or will or will not develop) one of several different diseases and disorders.

### BACKGROUND

With recent advances in large-scale sequencing technologies, it has become easier and more cost-effective to sequence the genomes or transcriptomes of individuals, allowing researchers to identify potential biomarkers for various diseases. Conventional biomarkers can include measurable variations in specific genetic loci or regions, e.g., the BRCA1 gene, variations in patterns on individual genes or genetic regions, e.g., changes in methylation patterns at CpG sites, changes is measurable analytes, e.g., creatinine, among others. Identifying conventional biomarkers associated with specific diseases or disorders can help in early detection, diagnosis, and treatment, as well as in developing personalized medicine approaches.

While conventional biomarker-based screening has the potential to revolutionize disease detection and prevention, there are several issues and limitations to consider. Testing and quantification of analyte biomarkers, for example, can be affected by timing, patient characteristics, and the like, such that a measured level may not directly reflect a health status of the patient. Moreover, an identified biomarker may not be specific to a particular disease or condition. For example, while the underlying mutation in Huntington's Disease is expansion of a CAG repeat in the Huntingtin (HTT) protein, there are hundreds of genes whose expression is dysregulated in patients with Huntington's Disease, and such changes have recently been found to depend on the disease duration, genetic background, and length of CAG repeat. Thus, a biomarker that is elevated in a Huntington's Disease patient could also be elevated in patients with other conditions or even in healthy individuals. The levels of certain biomarkers can vary widely within a population, and can be influenced by age, gender, genetics, and other factors. This variability can make it difficult to establish clear cutoff values for a given biomarker or set or biomarkers, which can in turn affect the sensitivity and specificity of the screening test.

### SUMMARY

The inventions disclosed herein overcome many of the limitations of prior art models by providing methods for classifying and predicting diseases and disorders using relational biomarkers. A relational biomarker is a mathematical function whose domain is two or more variables that represent patterns of or on biological materials such as methylation levels on DNA, DNA fragmentome signatures, RNA transcription levels, RNA splicing isoforms, DNA copy number variants, protein abundance and modification, metabolite profiles, and the like. Said differently, a relational biomarker is a mathematical function that represents the covarying behavior of two or more conventional biomarkers.

The inventions disclosed herein are based on the discovery by the present inventor that relational biomarkers, specifically, the parameters of the mathematical functions that relational biomarkers embody, are different in subjects who have different health conditions. For example, these parameters are different in subjects who will or will not give birth prematurely (FIG. 4) and they are different in subjects who have Parkinson's Disease and those who have Alzheimer's Disease (Table 3). Because relational biomarkers represent functional relationships among patterns of or on biological materials, and the parameters of these relationships can distinguish health conditions including having or not having a disease or disorder, the current disclosure demonstrates that relational biomarkers characterize normal and abnormal system behaviors.

Accordingly, the present disclosure provides methods to discover relational biomarkers that distinguish health conditions. The methods generally include modeling relationships among the patterns on two or more biological materials for biological samples from each of a plurality of subjects having each health condition. In a case-noncase contrast, patterns of or on biological materials are collected for samples from each of a plurality of subjects who have the disease or disorder to determine a case relational biomarker, and similarly for each of a plurality of subjects absent the disease or disorder to determine a noncase relational biomarker. Various implementations of the methods may include generating ensembles of case and noncase relational biomarkers by repeating the modeling for additional combinations of biological materials. Other implementations may include generating ensembles of case-noncase classifiers, each of which may include two or more relational biomarkers.

The present disclosure also provides methods of classifying an unknown or holdout sample from a subject to distinguish the sample, and thus the subject, as having (i) a disease or disorder, or likely to develop the disease or disorder in the future, or (ii) not having the disease or disorder. When classification of an unknown sample is ambiguous, comparisons against other relational biomarkers from an ensemble of case and noncase relational biomarkers may be used to confidently classify the sample. An ensemble of case and noncase relational biomarkers may help to uniquely distinguish a sample as case or noncase as well as from a different disease or disorder, i.e., a second case. For example, relational biomarkers can distinguish aneuploid pregnancies from normal pregnancies and also can distinguish one trisomy from another (Table 3). Ensembles of relational biomarkers can boost the accuracy of classifiers, and ensembles of classifiers can perform multiclass classification in which the task is to identify which of multiple health conditions is represented by a sample (see the section on Multiclass Classification).

The present disclosure further provides methods to discover discriminators (also known as models) that classify samples as having different health conditions. Discriminators comprise pairs of relational biomarkers. For example, a discriminator for a case-noncase contrast comprises a relational biomarker pair wherein the pair models mathematically a relationship between patterns of a first biological material and at least one additional biological material for each of the plurality of subjects having the disease or disorder to determine a case relational biomarker, and for each of the plurality of subjects not having the disease or disorder to determine a noncase relational biomarker.

In the present disclosure, biological sample(s) may comprise one or more biofluid or tissue samples of the subject. Moreover, the biological material(s) may be nucleic acid(s), protein(s), metabolite(s), or any combination thereof. When the biological material is nucleic acid, a pattern of the material may comprise a frequency of a state of the nucleic acid, such as methylation, gene expression, RNA expression, RNA isoforms, estimated fetal fraction of hypermethylated cell-free nucleic acid (cfNA), estimated fetal fraction of hypomethylated cfNA, and the like. Moreover, when the biological material is nucleic acid, the first and the at least one additional biological material may comprise: the same nucleic acid region from biological samples taken at different time points; regions of the same chromosome; regions of different chromosomes; a first and at least a second gene; a first and at least a second regulatory region; a first and at least a second transcribed gene; a first and at least a second region of a transcriptome; or any combination thereof.

Alternatively, or additionally, when the biological material is a protein, the state of the protein may comprise abundance or localization of one or more proteins, proteoforms, posttranslational modification(s), degradation state (e.g., fragment identity, localization, and/or abundance), or protein interaction partner(s). Moreover, the first region and the at least one additional biological material may comprise: different proteins, the same protein from biological samples taken at different time points, the same protein from different locations in a cell, organ, or organism, or any combination thereof.

Alternatively, or additionally, the biological material may include metabolites, i.e., low-molecular-weight molecules (metabolites) present in the cell that are participants in general metabolic reactions and that are required for the maintenance, growth, and normal function of a cell. When the biological material is a metabolite, the state of the metabolite may comprise patterns of these molecules, such as patterns of identity and/or concentrations of metabolites in a biological sample, measured as a function of time and/or location (e.g., cellular, tissue, or organ specific).

According to any of the aforementioned aspects, the first and the at least one additional biological material may be selected to maximize an estimated accuracy of classifiers based on discriminators, that is, pairs of relational biomarkers.

The present disclosure further provides one or more computer storage media having computer-executable instructions embodied thereon that, when executed, perform any of the disclosed methods.

The present disclosure further yet provides systems for distinguishing disease or disorder samples from noncase samples. The system generally comprises a server coupled to a wireless network and configured to communicate with a plurality of client devices. The server comprises a processor configured to execute instructions that perform any of the methods disclosed herein, and may be coupled to a database comprising multiomic data and/or variables that may be used by the processor, such as to (i) distinguish a sample as a disease or disorder sample or a noncase sample, (ii) discover relational biomarkers that discriminate disease or disorder samples from noncase samples, and/or (iii) model system behavior that distinguishes disease or disorder samples from a noncase samples.

The details of various embodiments are set forth in the accompanying drawings and the description below. Other features and advantages will be apparent from the description and drawings, and from the claims.

### BRIEF DESCRIPTION OF THE DRAWINGS

FIG. 1 illustrates a relationship among patterns on three different chromosomes according to models and methods of the present disclosure. All values are counts of patterns on genomic material that have been min-max scaled, that is, x_scaled=(x−min(X))/(max(X)−min(X)) for each x in distribution X.

FIG. 2 illustrates the relationship shown in FIG. 1, wherein samples from subjects having preterm birth pregnancies are shown as solid squares, while those from subjects having normal term birth pregnancies are shown as open circles. The frequencies of patterns in genomic material have been min-max scaled as specified in the description of FIG. 1.

FIG. 3 illustrates the functional relationship shown in FIG. 2 for chromosomes 9 and 1, wherein pregnancies can be modeled by mathematical functions with parameters b0 and b1 such that these parameters have different values in normal term and preterm birth pregnancies. The frequencies of patterns in genomic material have been min-max scaled as specified in the description of FIG. 1.

FIG. 4 illustrates parameters for relational biomarkers for subsamples drawn from normal term and preterm birth pregnancies.

FIG. 5 illustrates the frequencies of given patterns on chromosomes 9 and 1 for samples from normal term and preterm birth pregnancies. The frequencies have been min-max scaled as specified in the description of FIG. 1.

FIG. 6 illustrates a functional relationship between patterns on chromosomes 1 and 21 for Trisomy 21 and non-Trisomy 21 pregnancies. The variates have been min-max scaled as specified in the description of FIG. 1. The relational biomarkers for Trisomy 21 fit to the solid squares whose range is shown as the heavy line with an extrapolation to outside of the range shown as the dashed line, wherein the star shaped points are from a new sample outside this range.

FIGS. 7A and 7B illustrate classification of a sample (black dot) based on relational biomarkers determined using methods of the present disclosure, wherein FIG. 7A illustrates a situation when the sample appears near the intersection of relational biomarkers, and FIG. 7B illustrates use of another set of relational biomarkers, i.e., from an ensemble of relational biomarkers, with similar sensitivity and specificity in which the sample does not appear near their intersection.

FIG. 8 shows block diagrams for methods of discovering relational biomarkers and screening for diseases/disorders using relational biomarkers according to the present disclosure.

FIG. 9 illustrates classification of new samples (black dots) based on relational biomarkers determined using methods of the present disclosure. Given a new sample for which X and Y are known (large black dot), each relational biomarker can predict Y given X. The differences between the true Y and the ctrl_Y_predicted and between the true Y and case_Y_predicted are used to decide whether the new sample is a case or a noncase. This process can be repeated for subsamples from a sample (small black dots) to get a confidence score for the classification of the sample.

FIG. 10 shows a fragment of a table that illustrates the output of an ensemble of binary classifiers for a multiclass classifier. This classifier classifies samples as belonging to one of seven health conditions: Trisomies 13, 21 and 18 (not shown), Monosomy X, severe preeclampsia (pes), mild preeclampsia (pem) and preterm birth (preterm). Each column represents a biological sample, each row represents a binary classifier for a contrast A,B (e.g., tri21,pes). Each cell contains a number between +1, meaning the classifier favors A or −1, meaning the classifier favors B. The “Prediction” row represents the ensemble judgment of all the binary classifiers. The “Ground Truth” row represents the clinical characterization of the sample. The “Confidence” row contains numbers between 0 and +1 that represent the confidence of the ensemble in its prediction.

FIG. 11 illustrates a randomized distribution of the best accuracy Matthews Correlation Coefficient (MCC) scores from all possible pairs of relational biomarkers under the null hypothesis that cases and noncases do not have different statistics for their patterns.

FIG. 12 illustrates a block diagram of the hardware and software modules of the systems configured to perform the methods of the present disclosure.

### DETAILED DESCRIPTION

This disclosure relies on terms such as biological material, pattern, biomarker, model, classifier, ensemble, and others. Definitions of these terms are presented in Table 1 and are discussed and illustrated throughout this application. Many of the examples in this disclosure are familiar case-control, i.e., case-noncase, contrasts between subjects who have a disease or disorder and subjects who do not have the disease or disorder, but we note that the methods herein are more generally for distinguishing multiple health conditions (i.e., binary contrasts between two diseases and multiclass contrasts among several diseases).

The scope of this application includes three kinds of classification: case-noncase (AKA case-control), case-case, and multiclass. Case-noncase classification means inferring whether or not a subject has or will have a disease or disorder; case-case classification means inferring which of two diseases or disorders a subject has or will have; multiclass classification means inferring which of several health conditions, including diseases and disorders and also no disease or disorder, a subject has or will have. Throughout this application, case-noncase classification denotes a superclass that contains case-case classification. Of note, a noncase sample in one discriminator could be a case sample for a different disease or disorder in another discriminator.

TABLE 1

| | |
|---|---|
| Biological Material | Quantifiable amounts or proportions of material from |
| | different -omes, including nucleic acids, proteins(e.g., antibodies, |
| | enzymes, antigens, etc.), lipids, metabolites, or combinations thereof. |
| | Biological material may come from biofluids or tissue samples. |
| Region | In some cases, we can localize biological material to a region; for |
| | example, a fragment of cell-free DNA can be localized to a gene |
| | promoter region or a chromosome or a CpG island. |
| Pattern of Biological | A mathematical representation of the information contained in |
| Material | biological material. Examples include the raw amount of cell-free |
| | DNA, the distribution of genes lengths on a chromosome, the |
| | variance of the distance between CpG sites, the covariance of |
| | methylation of adjacent CpG sites, and so on. |
| Biological Sample | Biological material from one subject or from one or more |
| | “connected” entities such as mother and fetus, host, and tumor, etc. |
| Pseudosample | The result of randomly sampling biological material, or sampling |
| | from a pattern of biological material. For example, when the |
| | probability p of methylation within a region is known, a |
| | pseudosample of a region methylation profile can be obtained by |
| | sampling from a Binomial with probability p. |
| Condition | A clinical condition such as preeclampsia or Parkinson's disease. |
| Group | A batch of samples that share attributes such as conditions, -omes, |
| | lab procedures, etc .; for example, a batch of samples from Trisomy |
| | 21 pregnancies for which we have paired-end DNAseq data. |
| Cases, Noncases and | Subjects who have or will develop a given clinical condition may be |
| Controls | referred to as cases; subjects who don't have and do not develop the |
| | condition may be referred to as noncases or controls. |
| Conventional | Quantified patterns of biological material from one or more samples; |
| Biomarker | for example, the proportion of methylated CpG sites in a gene |
| | promoter region. |
| Relational Biomarker | A mathematical function over conventional biomarkers from samples |
| | or pseudosamples, typically assessed over groups, i.e., a |
| | mathematical function that represents the covarying behavior of two |
| | or more conventional biomarkers. |
| Model, AKA | Any mathematical formulation of contrasts, typically involving |
| Discriminator | ordinary or relational biomarkers, which may be used in a classifier. |
| Contrast | Any quantitative comparison of groups and by extension the |
| | conditions that characterize the groups; for example, subjects with |
| | Parkinson's Disease in contrast with subjects who have no |
| | neurological disease, or subjects with Parkinson's in contrast with |
| | subjects with Alzheimer's Disease. |
| Classifier | Any algorithmic procedure for assigning a sample to one or more |
| | conditions based on a contrast. For example, in a contrast of Trisomy |
| | 21 and normal pregnancies, a classifier would assign a sample to one |
| | condition or the other. Classifiers may also resolve contrasts between |
| | different disease or disorder conditions; for example, assigning a |
| | sample to Trisomy 21 or Trisomy 18. Some classifiers return “no- |
| | call;” for example, a classifier might not have sufficient evidence to |
| | differentiate one trisomy from another. Binary classifiers resolve |
| | contrasts of two conditions, multiclass classifiers resolve contrasts of |
| | more than two conditions. Some classifiers function as predictors in |
| | the sense that they classify a sample as likely to experience a |
| | condition in the future; for example, predicting that a baby will be |
| | born preterm based on a sample collected at the end of the first |
| | trimester. |
| Discovery of | Any algorithmic process that constructs models to maximize an |
| classifiers | objective function; for example, a process that constructs sparse |
| | models that are accurate, resistant to overfitting, and minimizes no- |
| | calls. |
| Ensemble of | A collection of ordinary or relational biomarkers that may be used in |
| biomarkers | a classifier. |
| Ensemble of | A collection of classifiers that may be used in a binary or multiclass |
| classifiers | classifier. |
| Ensemble classifier | Any classifier that uses an ensemble of biomarkers or (more |
| | commonly) an ensemble of classifiers. |
| New sample, held-out | A sample that is not used to train a classifier |
| sample, test sample | |

The inventions disclosed herein are based on the discovery of functional relationships between patterns on two or more biological materials (e.g., two or more regions of nucleic acid, proteins, metabolites, etc.) and the compensatory changes in those relationships in biological samples from subjects having a disease or disorder. That is, the present inventor has developed methods to model system behaviors based on functional relationships between measurable quantities, such as DNA methylation, gene expression, protein modification, and the like, on two or more biological materials.

To explain the present methods, we will describe compensatory changes using a simple model of a human driving a car. When a road curves left, the driver turns the steering wheel counterclockwise to the left, and when the road curves right, the driver turns the steering wheel clockwise to the right. When the road is straight, the driver makes only small adjustments but no large or sustained movements of the steering wheel. If we let X be the twists and turns in the road and Y be a driver's steering to track the road, we can describe this steering behavior by a function Y=F(X, θ), where θ=θ₁, θ₂, . . . , θₖ denotes the parameters of the function; for example, θ might describe how hard the steering wheel is turned to the left or right.

Should the left-front tire of a car be under-inflated, the car will pull to the left and the driver must compensate by turning the steering wheel to the right. The car's behavior might still be described by F, but with different parameters θ to represent the driver's compensatory steering. Thus, Y=(X, θ+) might describe a mathematical model of “case” samples of driving when the left front tire is under-inflated while Y=F(X, θ−) might be a mathematical model of “noncase” samples of driving with normally inflated tires.

Extending this analogy into biological systems, the present inventor has found that a pattern of one biological material, e.g., region of nucleic acid, is related to a pattern of a second, third, and additional biological material, e.g., additional regions of nucleic acid, such that a mathematical function Y=F(X₁, X₂, . . . , θ) describes the relationship. The symbols Y and X represent patterns on the biological material and O represents the parameters of the function F.

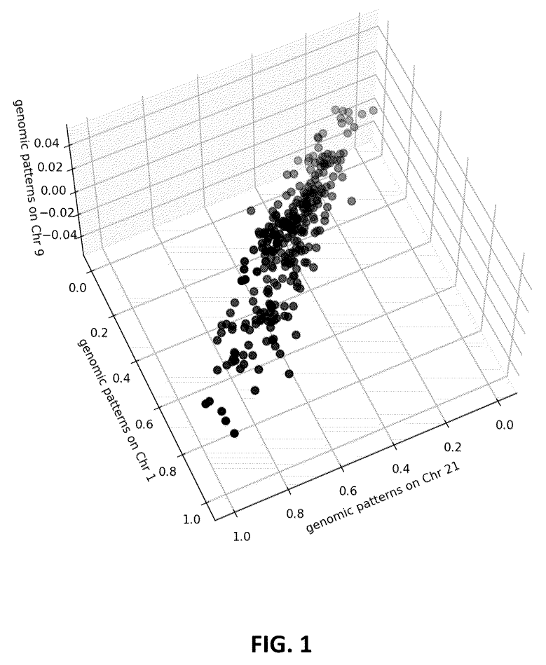

As example, and with reference to FIG. 1, we may construct a mathematical function that relates three conventional biomarkers Y, X₁ and X₂. In this example, Y represents the frequency of a pattern of methylation on chromosome 9 (Chr 9), X₁ represents the frequency of a pattern of methylation on chromosome 1 (Chr 1), and X₂ represents the frequency of a pattern of methylation on chromosome 21 (Chr 21). All values are counts of patterns on genomic material that have been min-max scaled, that is, x_scaled=(x−min(X))/(max(X)−min(X)) for each x in distribution X. Note that Y appears to be a roughly linear function of X₁ and X₂ such that an increase in the amounts of X₁ or X₂ is associated with an increase in Y. We can construct a mathematical function Y=β₀+β₁X₁+β₂X₂+ε to represent the relationship between Y, X₁ and X₂.

### Relational Biomarkers

The present disclosure relates to mathematical functions Y=F(X₁, X₂, . . . , θ) that describe how a pattern of one biological material is related to other patterns on a second, third, and additional biological material. The elements X and Y may be referred to as biomarkers and the function Y=F(X, θ) is referred to as a relational biomarker. That is, a biomarker that describes mathematically how conventional biomarkers are related. For example, if the relationship between the three kinds of biological material in FIG. 1 were linear, then a relational biomarker might be a plane in three dimensions. In some contexts, Y=F(X, θ) will be described as compensatory, meaning that the mathematical relationship between patterns or biomarkers X and Y represents a compensation by a biological organism to a condition.

For compactness we will denote relational biomarkers as F(R, θ), where we intend R to be a matrix of two or more genomic patterns in specific regions, that is, R=(Y, X₁, X₂, . . . ).

Note that R can be multi-omic; for example, X might be methylation patterns on Chromosome 1 while Y might be RNA transcripts of a particular gene. Relational biomarkers can be constructed for any patterns of or on biological materials, including hypo- and hyper-methylation, levels of gene expression, numbers of RNA transcripts, probability distributions over gene isoforms, protein distribution and/or abundance, metabolite profiles and abundance, and the like (see Table 3 for examples). Moreover, relational biomarkers can be constructed for more or less targeted biological materials, such as more or less targeted regions of nucleic acid, including entire chromosomes, gene promoter regions, gene exons, CpG islands, and the like.

As used herein, the phrases “pattern of” and “pattern on” a biological material are interchangeable unless specifically indicated otherwise. Thus, a pattern of a biological material should be understood to encompass not only a pattern of temporal or spatial expression, temporal or spatial abundance, temporal or spatial fragmentation, and the like, but also a pattern on a biological material such a pattern of temporal or spatial modification, such as methylation on DNA or posttranslational modification(s) on a protein, etc.

The present inventor has found that relational biomarkers for samples from a subject having a disease or disorder may have different parameter values than relational biomarkers for samples from a subject absent that disease or disorder. Said differently, a discriminator, or pair of relational biomarkers, might access the same patterns on biological materials but the covariation of this material will be different in cases and noncases. Accordingly, the methods disclosed herein comprise methods to discover relational biomarkers for a plurality of subjects having a disease or disorder as well as relational biomarkers for a plurality of subjects who do not have the disease or disorder (see the section “Discovery Algorithm”). Because they may have different parameter values, we denote these relational biomarkers as F(R, θ+) and F(R, θ−), for subjects having the disease or disorder and subjects who do not have the disease or disorder, respectively. (The same notation is used for case-case discriminations of two diseases or disorders, although here the denotation of one as + and the other as − is arbitrary.)

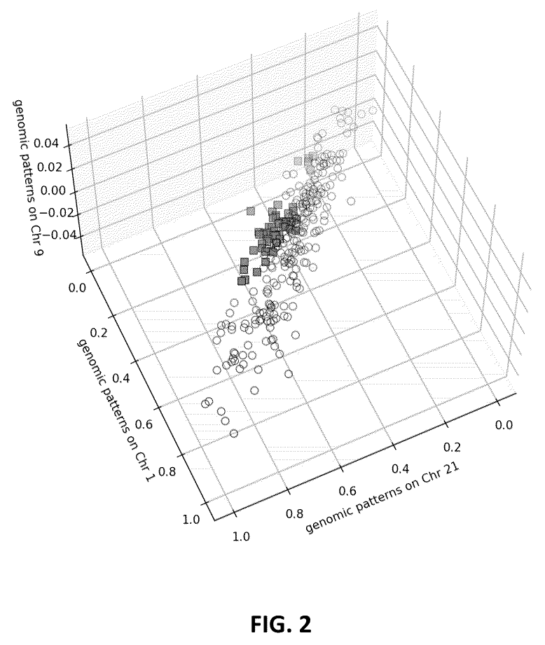

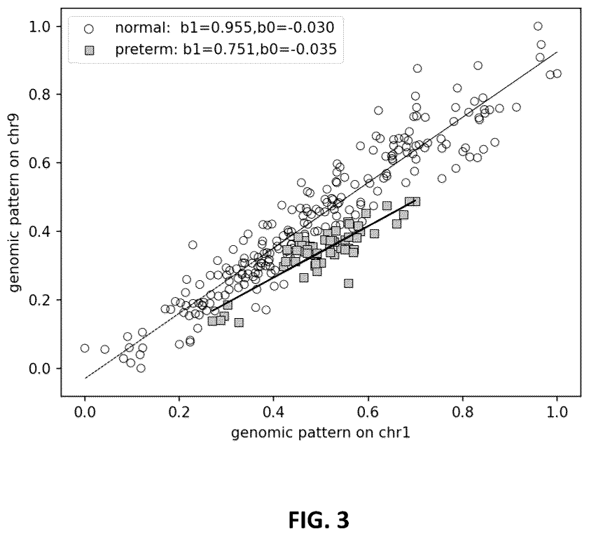

As another example of the methods disclosed herein, FIG. 2 shows the same data as in FIG. 1, except the symbols for the points are changed to distinguish samples of normal term pregnancies from preterm birth pregnancies. The solid square points represent samples obtained from pregnant women who will give birth prematurely while the open circle points represent samples from women who will have normal term births. It might not be apparent in FIG. 2 that the relational biomarker for preterm samples has different parameters than the relational biomarker for normal term samples, but this is clear in FIG. 3, which shows the same data projected onto two dimensions, resulting in two-dimensional relational biomarkers that relate methylation patterns on Chr 9 and Chr 1. The normal term pregnancy samples are modeled by a line that has different parameter values than the preterm birth samples. The differences between these parameter values are small, but they are sufficient to classify with high accuracy samples as coming from pregnancies that will later result in normal term or preterm birth. This is surprising because the samples were collected at the end of the first trimester, many weeks before birth. (For examples involving other -omes and diseases, see the section titled “Some representative results”).

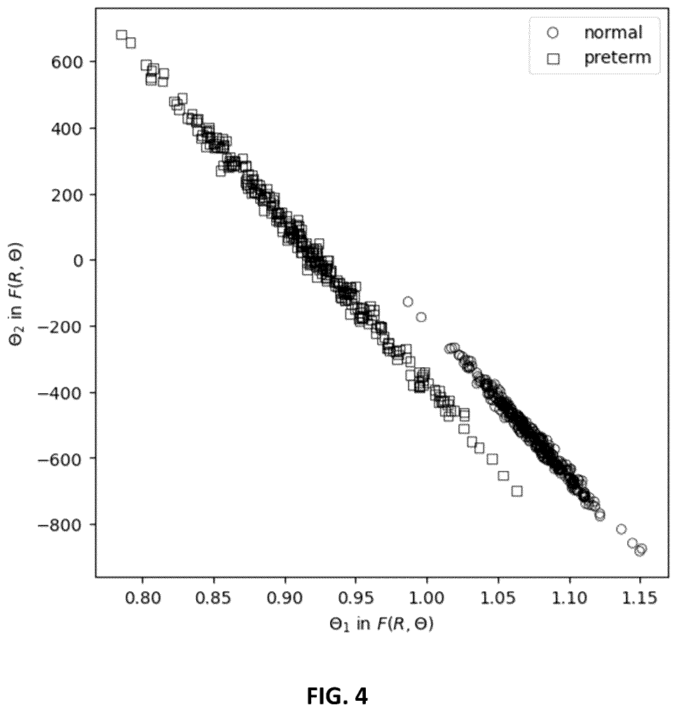

The functional form of relational biomarkers F(R, θ+) and F(R, θ−) may be identical but parameters θ+ and θ− may differ in different health conditions. This point is illustrated in FIG. 4, which shows parameter values for relational biomarkers fit to subsamples of methylation patterns on chromosomes 9 and 1 for preterm birth and normal term birth samples. Two points are notable: The parameter values θ+ for preterm relational biomarkers (squares) are highly separable from the parameter values θ− from normal term relational biomarkers (circles), and the variance of θ+ is higher than the variance of θ. We observe this difference in variance in many diseases: We believe it likely represents the body trying to compensate for disease.

### Advantages of Relational Biomarkers

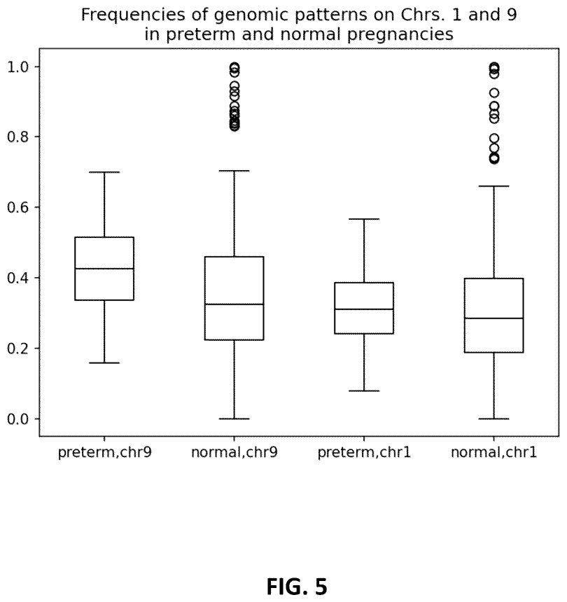

One strong advantage of relational biomarkers is that a relational biomarker F(R, θ) might be diagnostic or predictive when none of its constituent conventional biomarkers R=(Y, X₁, X₂, . . . ) is diagnostic or predictive. This point is illustrated by comparing FIG. 3 with FIG. 5. As noted, FIG. 3 shows that the relational biomarkers for frequencies of methylation patterns on chromosomes 9 and 1 have different parameter values. FIG. 5 shows these frequencies. Clearly, the frequencies of patterns on chromosome 9 are not very different in cases and noncases, nor are the frequencies of patterns on chromosome 1. In a conventional search for biomarkers, neither would qualify. Yet, FIG. 3 shows that these methylation patterns on chromosomes 9 and 1 are excellent relational biomarkers. Thus, the present methods allow identification of highly diagnostic mathematical relations between biomarkers even when none of the biomarkers is itself diagnostic. Said differently, the variance in FIG. 5 that obscures the difference between cases and noncases is actually an advantage if one can model mathematically the covariation of biomarkers, as we do in relational biomarkers.

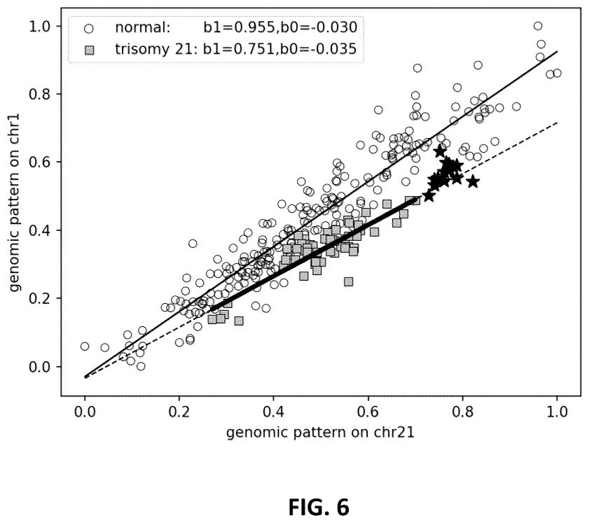

Relational biomarkers generalize well to previously unseen cases. Because relational biomarkers are mathematical functions, their ranges are not bounded by observed data. This is illustrated in FIG. 6, which shows relational biomarkers that accurately detect Trisomy 21 (Down Syndrome, Table 3). Note the observed range of patterns on chromosome 21 is relatively narrow for Trisomy 21 samples (squares, range is the darker line). New samples might fall outside this range, shown as the star shaped points in the range of the dashed line in FIG. 6, and be classified as noncase samples as they are within the broader range of the noncase samples (circles). However, the new samples are classified correctly as Trisomy 21 using the methods of the present disclosure because the relational biomarker for Trisomy 21 is a mathematical function whose range extends beyond the observed data (dashed line). This ability to generalize to new cases is not a feature of all machine learning classifiers. For example, if the prior art nearest-neighbor classifier were used, out-of-range new samples might be classified incorrectly. Thus, the presently disclosed methods provide models of system behavior that implicitly describe data not previously observed.

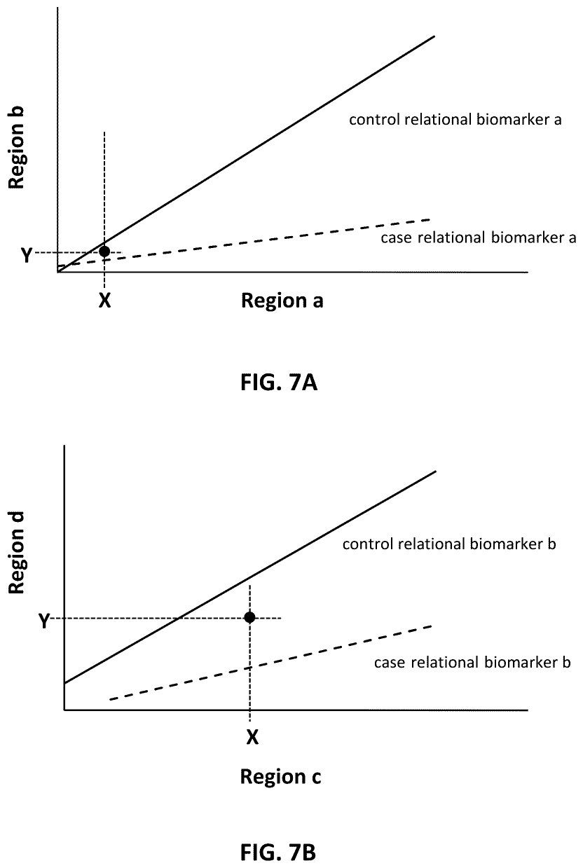

Further, should the new sample fall within a range where unambiguous classification is not possible, such as near the intersection of the case and noncase relational biomarkers (e.g., intersection of the two lines of FIG. 6, which is shown schematically in FIG. 7A), different discriminators may be used, i.e., selecting different sets of case and noncase relational biomarkers from an ensemble of relational biomarkers. As shown in FIGS. 7A and 7B, when a discriminator pair ‘a’, i.e., relational biomarkers a and b, does not provide unambiguous classification of a sample as case or noncase, a different discriminator pair ‘b’, i.e., relational biomarkers c and d, may be selected.

Another advantage of relational biomarkers is that they are sparse models and as such are robust against overfitting. A sparse model has few parameters relative to the number of data points that are used to estimate these parameters. Overfitting arises when the models have many parameters and few data. Overfitting is a significant problem for conventional biomarkers. For example, there are roughly 29 million CpG sites at which the human genome may be methylated, so it is virtually certain that methylation or lack of it at one or more of these sites will correctly classify virtually any disease given relatively few case samples and noncase samples. However, overfit models generalize poorly and often mis-classify new samples. To guard against overfitting, statisticians apply very stringent methods such as Bonferroni adjustments and Benjamini-Hochberg adjustments. But any adjustment that makes false discovery of an overfit classifier more difficult also makes discovery of a truly general classifier more difficult, so many conventional biomarkers that might be useful are discarded. Relational biomarkers rarely overfit data because they are sparse, that is, they have few parameters; for example, the relational biomarkers illustrated by the examples provided in this disclosure have only two parameters each, the slope and intercept of a line.

### Classification and Discovering Classifiers

Based on the discovery of relational biomarkers, the present inventor has developed an algorithm to classify new samples as cases and noncases with high accuracy (see ‘Classification Algorithm’ section). By “new” we mean samples that are not used to construct relational biomarkers for cases and noncases. The present inventor has also developed a Discovery algorithm to find relational biomarkers F(R, θ+) and F(R, θ−) that maximize the accuracy of classifying new samples (see ‘Discovery Algorithm’ section). The Discovery algorithm uses examples of cases and noncases to find the patterns and model parameters that distinguish new samples as case or noncase. That is, the Discovery algorithm searches for patterns R=X₁, X₂, . . . , Xₖ and model parameters θ+, θ− such that case and noncase relational biomarkers can distinguish new case samples from new noncase samples. The Discovery algorithm is multi-omic: Xᵢ might be methylation patterns in a region of the genome while Xⱼ might be RNA transcripts from an overlapping or nearby region.

### Workflows and Algorithms

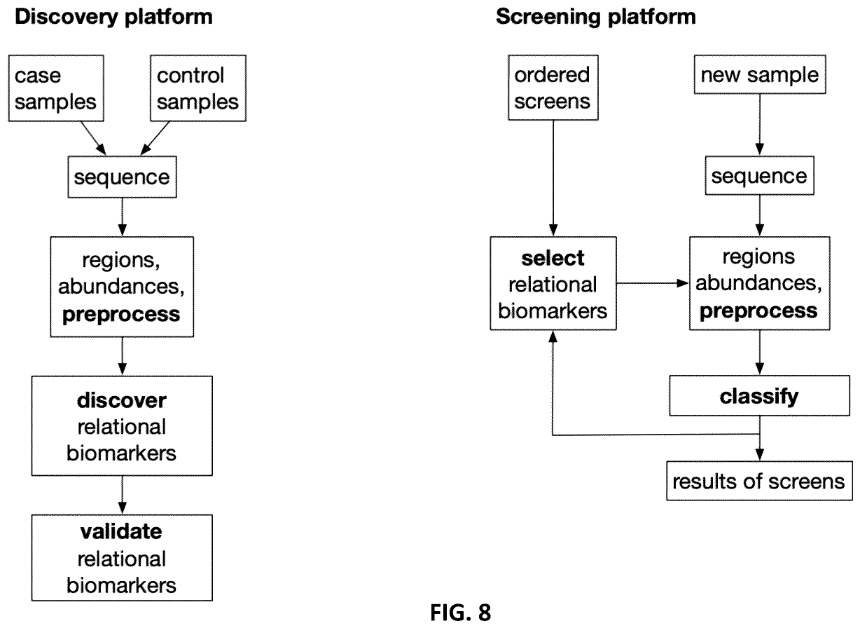

As such, the present disclosure provides two workflows or processes that discover relational biomarkers and uses them to classify new samples, respectively (FIG. 8). These workflows comprise steps that include data cleaning, the Discovery algorithm, the Classification algorithm, error analysis and power analysis, and the automated selection of panels of relational biomarkers to screen for multiple diseases or disorders. Data cleaning may involve pipelines to convert from one file format to another, imputation for missing data, scaling or other transformations of data, dealing with outliers and other possible errors, estimating generating distributions, and so on. Other steps in the workflows are described in the following paragraphs.

### Classification Algorithms

The present inventor has developed several classification algorithms for 1) binary case-noncase contrasts (i.e., saying whether a subject has a disease or not), 2) binary case-case contrasts (i.e., saying which of two diseases or health conditions is or will be present in a subject), 3) multiclass contrasts (i.e., saying which of more than two diseases or health conditions is or will be present in a subject). Further, these classification algorithms work with single samples, batches of pseudosamples, single discriminators (i.e., pairs of relational biomarkers), ensembles of discriminators, and ensembles of other classification algorithms. Further, all these algorithms may return a class label (e.g., Trisomy 21) or decline to return a label (a “no-call”).

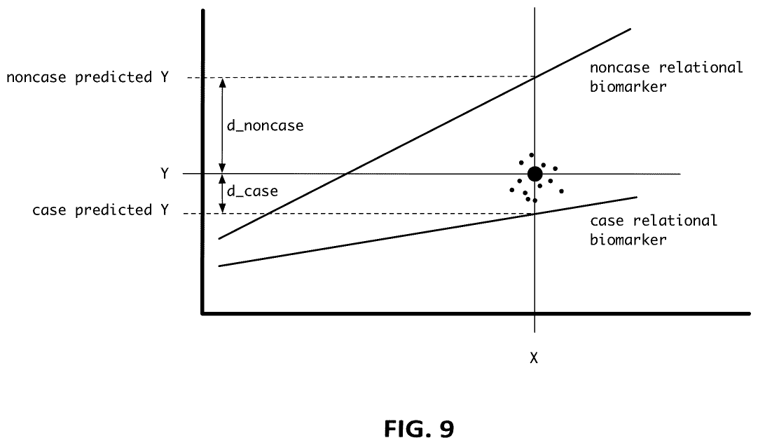

All these classification algorithms begin with one or an ensemble of discriminators. The discovery of good discriminators is discussed in the following section. Here we assume that a classifier has been given at least two relational biomarkers, one for cases, the other for noncases (alternatively, one for each of two diseases). We will assume-though only for expository purposes and without loss of generality-that each relational biomarker is a linear function that relates two patterns on biological materials, denoted X and Y, as shown in FIG. 9. That is, each relational biomarker plots Y as a linear function of X and the parameters of these functions are different; for example, the relational biomarker for noncases has a higher slope and lower intercept than the case relational biomarker. A new sample will have values for X and Y that plot as the large black dot in FIG. 9. The job of a classifier is to say whether the new sample is a case or a noncase, that is, whether the sample comes from a subject who does or does not have a disease or disorder.

Given the X value of a new sample, the case and noncase relational biomarkers can predict the Y value of the sample. This is a simple matter of finding the value on the Y axis at which a vertical line drawn through X intersects each relational biomarker line. A classifier asks whether the actual Y value of the new sample is closer to the value predicted by the case relational biomarker or the noncase relational biomarker. The deviations between the actual value of Y and those predicted by the case and noncase relational biomarkers are called d case and d_noncase, respectively, in FIG. 9. The simplest classifier says the new sample is a case if d case<d_noncase and conversely says a new sample is a noncase if d_noncase<d_case. A “no-call” is returned if the difference between d_case and d_noncase is small.

In practice, this simple classifier works very well (it is used for most conditions in Table 3). However, when a new sample contains a multiplicity of patterns on biological materials, or when it can be characterized by probability distributions over such patterns, then we can create pseudosamples of the sample by resampling from its multiple patterns or probabilities. Pseudosamples are shown as small black points in FIG. 9. Each pseudosample may then be classified by the simple classifier described in the previous paragraph. The class label of the sample itself is a function of the fraction of pseudosamples that are classified as case or noncase. If most or all pseudosamples are classified as cases, or at least a threshold proportion of pseudosamples are classified as case, the sample is classified as a case (and conversely for noncase). A “no-call” is returned if the proportions of pseudosamples classified as case and noncase respectively are similar, or subthreshold proportions may be used to classify samples as “no-call”. For example, the biological sample may be classified as: (i) case if a proportion of pseudosamples greater than (1-T), where T is a threshold, is so classified, or (ii) control if less than a proportion T of pseudosamples is so classified, or (iii) no-call if the proportion lies between T and (1-T).

A further refinement involves using not a single discriminator (i.e., pair of relational biomarkers) but an ensemble thereof. The reasons for doing this are discussed above and illustrated in FIGS. 7A and 7B. In practice, ensembles of discriminators “vote” on class labels for new samples.

Clearly, there is no functional difference between case-noncase contrasts and case-case contrasts in which the goal is to classify a new sample as one of two diseases.

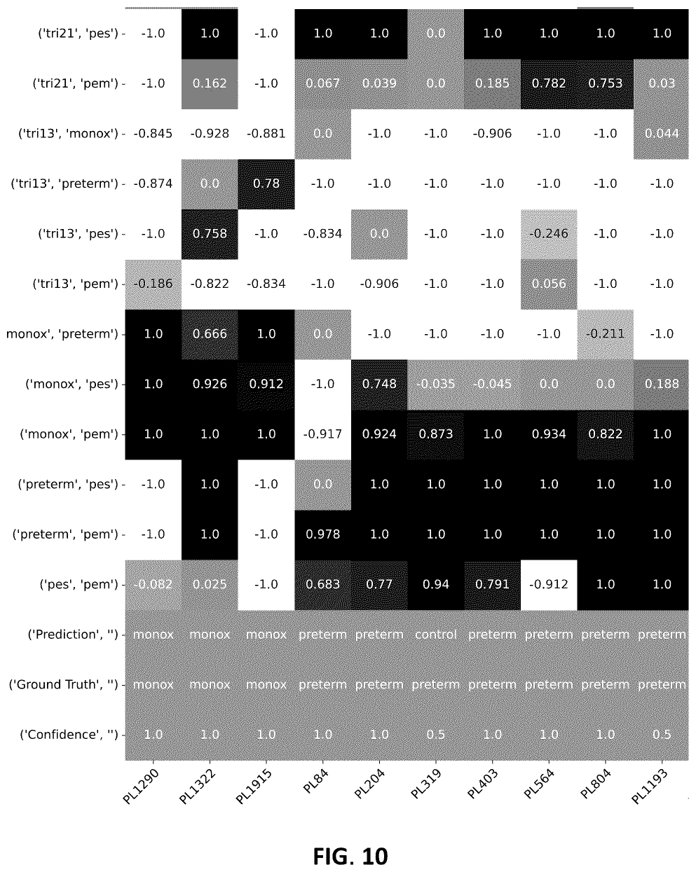

However, multiclass contrasts are quite different from binary contrasts. Here the problem is to say which of several diseases or disorders is present in a sample, keeping in mind the adage that “a patient may have as many diseases as they pleases.” The multiclass setup is illustrated in FIG. 10, which shows a fragment of an ensemble of binary classifiers. (The full ensemble contains 28 classifiers.) The columns in this figure represent samples, the rows represent classifiers of the kind we have been discussing: binary classifiers that distinguish cases from controls or one disease from another. Said differently, the rows represent an ensemble not of relational biomarkers but of classifiers. From top to bottom we see case-case classifiers (Trisomy 21 vs. severe preeclampsia, Trisomy 21 vs. mild preeclampsia . . . severe vs. mild preeclampsia). The numbers in the cells represent the strength of evidence for either the first or the second class label in a classifier. For example, the monosomy X vs. preterm birth classifier favors monosomy X in the first three samples (with scores of 1.0, 0.667 and 1.0, respectively). These are in fact monosomy X samples. It favors preterm birth (giving scores of −1) to samples 204, 319, 403, 564 and 1193; these are in fact preterm birth samples. For samples 84 and 804, this single binary classifier returns ambiguous scores (0 and −0.211, respectively) but the weight of evidence from other classifiers in the ensemble results in a prediction of preterm birth, which is correct. The only error in the table is calling sample 319 a control when it is preterm, although here the confidence score of 0.5 flags the sample as one that may require further attention.

Three multiclass methods all give highly accurate results: First, the weight of evidence for each class label is summed across each column. Second, a random forest classifier is made to treat the numbers in each column as features. Third, a neural network classifier can be made to treat each column of numbers as a feature vector. Other methods are possible and within the scope of the present disclosure.

Random forest and neural network classifiers require many training instances, many more than the eight samples shown in FIG. 10. However, as noted earlier, some kinds of patterns on biological material allow us to “gen up” samples to produce sufficient numbers of pseudosamples for these data-hungry ensemble classifiers.

### Discovery Algorithm

The current inventor has developed a Discovery algorithm to discover relational biomarkers that not only do a good job classifying samples that have or will have a given disease or disorder but are statistically likely to do a good job on new samples, previously unseen by classifiers. For example, methylation profiles on Chr 1 and Chr 21 do a good job of classifying Trisomy 21 (FIG. 6), while other biological materials and patterns may be better at predicting preterm birth (e.g., methylation patterns on Chr 1 and Chr 9, FIG. 3).

For any given disease or disorder, the Discovery algorithm finds and ranks many pairs of relational biomarkers, i.e., many discriminators. Sometimes these discriminators have comparable accuracy; for example, the top ten discriminators for distinguishing preterm birth cases from controls are shown in Table 2.

TABLE 2

| Disc | MCC | SENS | SPEC | TP | FN | FP | TN | caseR² | ctrlR² | LOO | Score |
|---|---|---|---|---|---|---|---|---|---|---|---|
| 0 | 0.793 | 0.875 | 0.933 | 14 | 2 | 3 | 42 | 0.914 | 0.970 | 0.793 | 0.725 |
| 1 | 0.716 | 0.938 | 0.844 | 15 | 1 | 7 | 38 | 0.914 | 0.970 | 0.716 | 0.654 |
| 2 | 0.738 | 0.750 | 0.956 | 12 | 4 | 2 | 43 | 0.876 | 0.967 | 0.738 | 0.647 |
| 3 | 0.661 | 0.750 | 0.911 | 12 | 4 | 4 | 41 | 0.898 | 0.973 | 0.661 | 0.593 |
| 4 | 0.639 | 0.875 | 0.822 | 14 | 2 | 8 | 37 | 0.916 | 0.974 | 0.639 | 0.585 |
| 5 | 0.639 | 0.875 | 0.822 | 14 | 2 | 8 | 37 | 0.916 | 0.974 | 0.639 | 0.585 |
| 6 | 0.666 | 0.875 | 0.844 | 14 | 2 | 7 | 38 | 0.915 | 0.970 | 0.639 | 0.584 |
| 7 | 0.676 | 0.812 | 0.889 | 13 | 3 | 5 | 40 | 0.848 | 0.963 | 0.676 | 0.573 |
| 8 | 0.710 | 0.812 | 0.911 | 13 | 3 | 4 | 41 | 0.898 | 0.974 | 0.627 | 0.563 |
| 9 | 0.695 | 0.875 | 0.867 | 14 | 2 | 6 | 39 | 0.871 | 0.960 | 0.645 | 0.562 |

Abbreviations: Disc - discriminator or pair of relational biomarkers;
MCC - Matthews correlation coefficient, which is a correlation coefficient between the true and predicted classes, and achieves a high value only if the classifier obtains good results in all the entries of a confusion matrix, i.e.,
$M = ( \begin{matrix}TP & FN \\ FP & TN\end{matrix} ) ;$ SENS - Sensitivity, which denotes the rate of positive samples correctly classified, and is calculated as the ratio between correctly classified positive samples and all samples assigned clinically to the positive class;
SPEC - Specificity, which denotes the rate of negative samples correctly classified, and is calculated as the ratio between correctly classified negative samples and all samples assigned clinically to the negative class;
TP - True Positive, which denotes the number of correctly classified positive samples;
FN - False Negative, which denotes the number of samples incorrectly classified as negative;
FP - False Positive, which denotes the number of samples incorrectly classified as positive;
TN - True Negative, which denotes the number of correctly classified negative samples;
CaseR² and CtrlR² - coefficient of determination for case and control relational biomarkers, respectively, which is a statistical measure that represents the proportion of the variance for a dependent variable that's explained by an independent variable; and
LOO - Leave One Out, which denotes a cross validation method where a single sample is iteratively left out to define a test set with the remaining samples being the training set.
Score - a weighted sum of some of the preceding measures.

The sensitivities and specificities of these discriminators fall between 0.75 and 0.938 and between 0.822 and 0.956, respectively. The number of true positive classifications of 15 preterm birth samples ranges from 12 to 15, while the number of correct classifications of 45 control samples ranges from 37 to 43. The fact that many pairs of relational biomarkers perform comparably in case-noncase classifications has positive and negative consequences: Positive because several discriminators can be combined to make powerful ensemble classifiers as described in the previous section and FIGS. 7A, 7B, and 10; Negative because it is difficult to choose between apparently similar discriminators. For this latter reason, the Discovery algorithm combines several attributes of relational biomarkers into an overall score for each pair of relational biomarkers. These attributes estimate a classification accuracy on as-yet unseen samples. Three such criteria are caseR2, ctrlR2 and LOO. The first two evaluate how well each relational biomarker fits the samples on which it is trained. A poor fit suggests that future samples won't be well-represented by the relational biomarkers. The LOO criterion stands for “leave one out” accuracy. LOO accuracy is estimated by testing each sample in a training set with discriminators that have never seen that sample. Other criteria, not shown in the table above, include the proportion of cases that are classified as no-calls and confidence intervals associated with the relational biomarker parameters. All of these are combined into a single score for each discriminator. The highly efficient Discovery algorithm evaluates thousands and sometimes millions of relational biomarkers searching for those that have high scores.

### Validation of Relational Biomarker-Based Classifiers

The Discovery algorithm searches for relational biomarkers that promise highly accurate discriminations between case and noncase samples. The algorithm implements leave-one-out and other estimators within its inner loop to ensure unbiased estimates of accuracy on as-yet unseen samples. The accuracy figures in Table 3 are the results of classifying held-out samples. Thus, they are the best estimates of the accuracies that can be expected from classifying completely new samples.

However, because the Discovery algorithm searches very large numbers of pairs of relational biomarkers, there is a possibility that it will find relational biomarkers that respond to entirely accidental arrangements of data that have nothing to do with real differences between samples. It is necessary to demonstrate that when there is no difference between samples (e.g., between cases and noncases), the Discovery algorithm will not find high-scoring relational biomarkers that seem to distinguish between samples.

The present inventor has developed a randomized algorithm for estimating the accuracies of relational biomarker classifiers in the situation where cases and controls are not really different. All accuracy measures are arithmetic combinations of four numbers: TP is the number of true positives (i.e., cases classified as cases); FN is false negatives (cases not classified as cases); TN is true negatives (i.e., controls classified as controls); and FP is false positives (i.e., controls classified as cases). Sensitivity and specificity are TP/(TP+FN) and TN/(TN+FP), respectively. Sensitivity and specificity are combined in the Matthews Correlation Coefficient:

$$MCC = ( ( TN * TP ) - ( FP * FN ) ) / ( ( ( TN + FN ) * ( FP + TP ) * ( TN + FP ) * ( FN + TP ) ) ** 0.5 ) .$$

The randomized algorithm follows: Let N_case be the number of case samples and let N_noncase be the number of noncase samples. Pool the case and noncase samples in a “bucket” of samples. Repeat K=1000 times: 1) Shuffle the samples in the bucket; 2) Draw N_case samples from the bucket and call these case* samples and draw N_noncase samples from the bucket and call these noncase* samples; 3) Run the Discovery algorithm over all possible pairs of relational biomarkers to find the pair that differentiates the case* and noncase* samples with the highest accuracy (i.e., MCC score); 4) Record the MCC score obtained by the best pair of relational biomarkers discovered by the algorithm; and 5) Find the 99th quantile of the distribution of MCC scores. This will serve as a threshold for MCC scores from actual case-noncase tests.

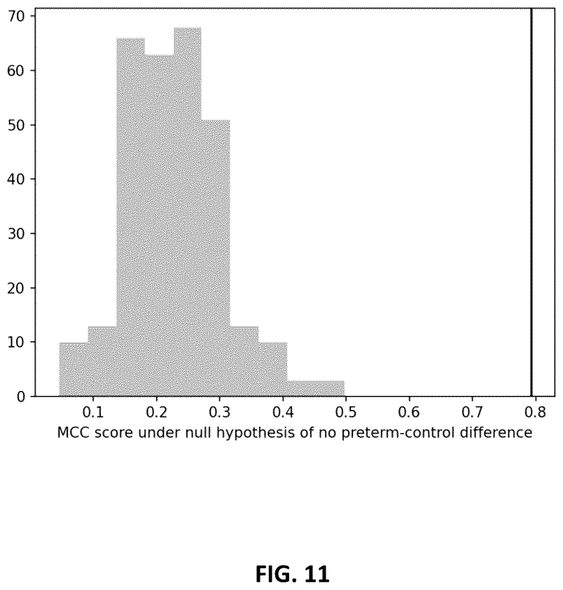

As an example, the randomized algorithm was run on the preterm birth samples and noncase samples shown in FIG. 3. The resulting distribution of K=1000 MCC scores is shown in FIG. 11. Each of these scores is the accuracy of the best of more than 2000 pairs of relational biomarkers in the condition where cases and noncases are not different, a condition enforced by throwing cases and noncases into a bucket and repeatedly drawing samples from the bucket and calling some samples case* and others noncase*, at random. Under this condition, the distribution of MCC scores, shown in FIG. 11, has a maximum value of slightly less than 0.5. In contrast, Table 2, above, shows the best ten MCC scores from a test for preterm birth when actual preterm birth cases are tested. All the MCC scores are well above 0.5. That is, each of the top ten discriminators in Table 2 produces MCC scores that would (probably) never arise if the Discovery algorithm were detecting accidental differences between preterm cases and noncases. This kind of validation by randomization tells us that the Discovery algorithm does not discover relational biomarkers that detect differences between cases and noncases when no such differences exist.

### Liquid Biopsies and Noninvasive Early Detection of Silent Diseases

Because relational biomarkers represent patterns on biological material that may be obtained from samples of blood, urine, stool, saliva, and other liquids, collectively known as “liquid biopsies,” they can diagnose or predict disease or disorders in the absence of clinical signs and symptoms. Consequently, these methods may detect or predict the emergence of so-called silent diseases that progress without expressing clinical signs and symptoms. Among the many silent diseases are neurodegenerative diseases such as Parkinson's Disease and Alzheimer's Disease, muscular-skeletal diseases such as osteoporosis, various cancers, and gestational syndromes such as preterm birth and preeclampsia.

The biological materials used in the methods of the present disclosure generally comprise nucleic acids, proteins, and/or metabolites. For example, when the biological material is nucleic acid, it may comprise sequence reads derived from biological samples of a subject or plurality of subjects. The sequence reads may be from a database or may be acquired from a sequencing device (e.g., see Table 3). Moreover, the nucleic acids may be selected as a subset of the plurality of nucleic acid sequence reads, wherein the subset may be curated or random. The methods may be repeated with various different subsets of the nucleic acids.

### Relational Biomarkers Based on Cell-Free Nucleic Acid Fragments

While sequence reads from any nucleic acid may be used in the disclosed methods, cell-free nucleic acids (cfNA) and/or mixtures of cfNA and cell-free fetal NA (cffNA) have several advantages. Circulating cfNA refers to fragments of NA that are released into the bloodstream through active secretion or from dying cells, including tumor cells. The cell-free nucleic acids useful in the methods of the present disclosure include both cell-free DNA (e.g., cfDNA, mixtures of cfDNA and cffDNA) and cell free RNA (e.g., cfRNA, mixtures of cfRNA and cffRNA). By sequencing these fragments and processing their statistics with the methods disclosed here, clinicians can gain insights into the genetic changes occurring in a patient's disease or disorder, as well as track its progression and monitor treatment response. Genomic sequencing of circulating cfDNA and/or cfRNA is a non-invasive approach to detecting and monitoring genetic alterations in cancer and other diseases. Further, the nucleic acid may be DNA, such as cell free DNA (cfDNA), a combination of cfDNA and cell free fetal DNA (cffDNA), or genomic DNA (gDNA), such as from tissue samples, wherein the cfDNA can comprise both nuclear cfDNA (n-cfDNA) and mitochondrial cfDNA (mt-cfDNA), as well as circulating tumor DNA (ctDNA) and donor-derived cfDNA (dd-cfDNA). The nucleic acid may also be RNA, such as cell free RNA (cfRNA), or a combination of cfRNA and cell free fetal RNA (cffRNA), wherein cell free RNAs can comprise mRNA, IRNA, tRNA, snRNA, siRNA, snoRNA, miRNA, circRNA, lncRNA, endoRNA, YRNA, and the like.

By the end of the first trimester, approximately 10% of the total circulating cell-free DNA in maternal plasma is of fetal origin (i.e., cffDNA), which has allowed development of non-invasive prenatal testing (NIPT) methods for detecting aneuploidy and other genetic alterations. It is common in current NIPT methods to use some level of enrichment of the cffDNA in the sample, such as physical enrichment and/or enrichment via analysis of fragment length, and estimates of the fetal fraction, that is, the proportion of all cell-free DNA that is cffDNA. Moreover, commercial NIPT methods generally do not provide functional or phenotypic insights into the health of the pregnancy and therefore cannot screen for complex diseases of gestation such as spontaneous preterm birth and preeclampsia.

The methods of the present disclosure do not require any enrichment of the cffDNA in a sample; nor do they require deconvolution of samples into fetal and maternal fractions of cffDNA and cfDNA; nor do they require pure tissue samples from which to derive reference data against which to compare circulating cell-free DNA fragments. Despite being simpler, more parsimonious, and making fewer assumptions, the methods of the present disclosure have been found to accurately provide functional insights into several gestational diseases or disorders, such as spontaneous preterm birth (FIGS. 3 and 4), preeclampsia, trisomies (e.g., trisomy 21, 18, 13), monosomies (i.e., monosomy X), bronchopulmonary dysplasia, and gender (see Table 3 and section “Some Representative Results”).

### Relational Biomarkers Based on Other Biological Material

Other biological material useful in the methods of the present disclosure may include proteins, such as proteins extracted from liquid biopsies and/or tissues. Such protein-containing samples may be analyzed, such as by determining the mass spectrometry profile in a sample of one or more normal species or stressed species, such as selected from derivatized, truncated, oxidized, methylated, deaminated, aggregated, differentially glycosylated, improperly disulfide bonded, or structurally intact protein species, fragments thereof, and contaminants therein. For example, standard mass spectrometry analysis data is generated by fragmenting or chemically modifying the reference biologic either before or while subjecting the sample to mass spectrometric analysis. While mass spectrometry analysis for protein-containing samples is common, other methods exist, such as nuclear magnetic resonance, protein microarrays, two-hybrid screening, western blots, and the like, and are constantly emerging.

Thus, when the biological material is a protein, the state of the protein may comprise abundance or localization of one or more proteins, proteoforms, posttranslational modification(s), degradation state (e.g., fragment identity, localization, and/or abundance), or protein interaction partner(s). Moreover, the first region and the at least one additional biological material may comprise: different proteins, the same protein from biological samples taken at different time points, the same protein from different locations in a cell, organ, or organism, or any combination thereof.

The biological material may also include metabolites, i.e., low-molecular-weight molecules (metabolites) present in the cell that are participants in general metabolic reactions and that are required for the maintenance, growth, and normal function of a cell. Specific signatures, i.e., patterns, of these molecules may define different diseases, disorders, or conditions. Various methods exist to measure metabolite profiles and concentrations in a biological sample, such as at least mass-spec, liquid chromatography, thin layer chromatography, ultrahigh performance liquid chromatography-mass spec, Fourier Transform Infrared Spectroscopy (FTIR), and nuclear magnetic resonance. Typical analysis of the metabolome involves use of combinations of these methods. Some of the metabolites analyzed may include, but are not limited to, those noted in The Human Metabolome Database (Wishart D S, et al., HMDB 5.0: the Human Metabolome Database for 2022, Nucleic Acids Res (2022) 7; 50(D1): D622-31).

Many diagnostic methods evaluate whether a biomarker is present or absent, or whether the “level” of a biomarker exceeds a threshold. The presently disclosed methods focus instead on mathematical relationships between biomarkers. As illustrated in FIGS. 3 and 5, these relational biomarkers may be diagnostic when none of their constituent biomarkers are.

While the disclosed and illustrated examples of the methods show comparisons of two biological materials, the method works equally well with higher-order relational biomarkers; say, three or more biological materials, e.g., chromosomes or regions of nucleic acid, metabolites from three or more time points or tissues, and the like. However, to avoid overfitting and loss of generalization, ensembles of low-order (i.e., sparse) relational biomarkers are practically preferred to higher-order relational biomarkers. Similarly, while the Discovery method can find non-linear relational biomarkers, linear models are preferred for their computational efficiency. In sum, relational biomarkers can be of any functional form, patterns can be drawn from biological material selections of any size (e.g., three or more chromosomal regions, three or more proteins, etc.), and the constituent biomarkers in a relational biomarker can be multi-omic.

### Some Representative Results

The Discovery Algorithm: To illustrate the power of relational biomarkers and the Discovery algorithm, we present results for several diseases and disorders (Table 3). In some cases, the Discovery algorithm was provided with case and noncase samples of patterns in different biological materials, e.g., genomic regions. In other cases, the algorithm discovered relational biomarkers to differentiate conditions; for example, differentiating Parkinson's Disease from Alzheimer's Disease.

In some diseases/disorders, the patterns on biological materials represent epigenetic factors, while in others the patterns represent gene expression. Other examples include relational biomarkers involving proteins or metabolites.

Although it would be easy to modify the parameter that controls the tradeoff between true and false positives (see discussion of “no-calls” in section “Classification Algorithms”), the following analyses were run with a fixed value of that parameter to emphasize that a single algorithm can discover relational biomarkers for multiple diseases and disorders without modification. The algorithm not only discovers relational biomarkers, but automatically ranks them. We report on the accuracy of classification using the highest-ranked relational biomarkers.

TABLE 3

| | | | | Type of genomic |
|---|---|---|---|---|
| | Condition | Sensitivity | Specificity | pattern |
| 1 | Trisomy 21 | 1.0 | 0.91 | methylation |
| 2 | Trisomy 18 | 1.0 | 0.93 | methylation |
| 3 | Monosomy X | 1.0 | 0.73 | methylation |
| 4 | Preterm Birth | 1.0 | 0.87 | methylation |
| 5 | Preeclampsia | 0.95 | 0.64 | methylation |
| 6 | Sex (male vs | 1.0 | 1.0 | methylation |
| | female) | | | |
| 7 | Bronchopulmonary | 0.92 | 0.85 | gene expression |
| | dysplasia | | | (DNA microarray) |
| 8 | smokers vs. | 0.81 | 0.74 | transcriptome |
| | nonsmokers with | | | (RNAseq) |
| | lung cancer | | | |
| 9 | Lung tumor tissue vs | 1.0 | 1.0 | transcriptome |
| | nonmalignant lung | | | (RNAseq) |
| | tissue | | | |
| 10 | Alzheimer's Disease | 0.91 | 0.95 | transcriptome |
| | | | | (RNAseq) |
| 11 | Parkinson's Disease | 0.94 | 0.77 | transcriptome |
| | | | | (RNAseq) |
| 12 | Alzheimer's vs. | 0.91 | 0.88 | transcriptome |
| | Parkinson's | | | (RNAseq) |
| 13 | Bacterial cause | 0.92 | 0.89 | metabolome NMR |
| | sepsis vs controls | | | spectra |
| 14 | Viral case sepsis | 0.81 | 0.87 | metabolome NMR |
| | vs controls | | | spectra |
| 15 | Viral vs Bacterial | 0.77 | 0.85 | metabolome NMR |
| | causes of sepsis | | | spectra |
| 16 | Late-onset | 1.0 | 1.0 | proteome |
| | | | | (data-dependent |
| | Preeclampsia | | | acquisition mass |
| | | | | spectrometry) |
| 17 | Early-onset | 0.82 | 1.0 | proteome |
| | | | | (data-dependent |
| | Preeclampsia | | | acquisition mass |
| | | | | spectrometry) |
| 18 | Canine Lymphoma | 1.0 | 1.0 | transcriptome |
| | | | | (RNASeq) |
| 19 | Canine Lymphoma | 1.0 | 1.0 | transcriptome |
| | in dogs with | | | (RNASeq of |
| | chemotherapy, | | | fine-needle aspirate |
| | early vs. late | | | samples) |
| | relapse | | | |
| 20 | Spina Bifida | 0.95 | 0.94 | methylation |
| | | | | (beadchip) solid |
| | | | | tissue biopsy |
| 21 | Anencephaly | 1.0 | 0.94 | methylation |
| | | | | (beadchip) solid |
| | | | | tissue biopsy |
| 22 | Preeclampsia cases: | 0.69 | 0.82 | cfRNA |
| | 2 genes 2 timepoints | | | (Illumina NovaSeq) |
| 23 | Preeclampsia: | 0.81 | 0.78 | cfRNA |
| | 1 gene 2 timepoints | | | (Illumina NovaSeq) |

Sensitivity and specificity are TP/(TP + FN) and TN/(TN + FP), respectively (TP—True Positive, FN—False Negative, FP—False Positive, and TN—True Negative).

Table 3 shows sensitivity and specificity measures for screens for three fetal aneuploidies (Trisomy 21, Trisomy 18, Monosomy X), for fetal sex, for preterm birth pregnancies, and for pre-eclampsia (rows 1-6 of Table 3). For these screens, the patterns on biological material collected from maternal plasma were specific patterns of methylation on cell-free DNA fragments from specific chromosomes (e.g., for preterm birth these chromosomes were Chr 1 and Chr 9). All samples were collected at the end of the first trimester. While it is not surprising that we can detect aneuploidies and sex chromosome anomalies early in pregnancy, it is remarkable that we can predict preterm birth and preeclampsia so early. To the best of our knowledge, these accuracy results are comparable to or better than those of published assays.

Table 3 also shows results for neonatal bronchial dysplasia (row 7), for which the patterns on biological material were the expression levels of particular genes as measured by DNA microarray technology.

Relational biomarkers can also be constructed from proteomic and metabolomic data. Table 3, rows 13-15 shows an analysis of relational biomarkers acquired from public metabolomic data from a study of sepsis in children (Grauslys, et al., NMR-based metabolic profiling provides diagnostic and prognostic information in critically ill children with suspected infection, Sci Rep (2020) 10:20198). Notably, the Discovery algorithm found many of the individual metabolites that were commented on by the study authors. For example, 3-hydroxybuterate, which was one of the strongest signals in the original study, was the second-strongest relational biomarker in our analysis. Interestingly, its partners were 2-hydroxyvalerate, histidine, lysine, and lactate (recall that all relational biomarkers relate at least two conventional biomarkers). Of these, the authors identified lactate as a moderately strong biomarker, but found no signal from histidine, lysine or 2-hydroxyvalerate. This reminds us of the example in FIG. 5, which shows that relational biomarkers can be strongly predictive even when constituent conventional biomarkers provide weak signals.

As another example, the most predictive metabolite in our analysis was glutamate paired with 2-hydroxyvalerate or with phenylalanine or valine, of which the authors called out the latter two as strong predictors. Thus, our first-ranked metabolite is associated with two that the authors identified and 2-hydroxyvalerate, while our second-ranked metabolite is associated with one that the authors identified and with 2-hydroxyvalerate. Among relational biomarkers, 2-hydroxyvalerate contributes a strong signal; among conventional biomarkers it does not. As a final example, in discriminating virally caused sepsis from controls, the authors found a weak association with citrate. In our analysis it appeared six times in an ensemble of the best 20 relational biomarkers. Thus, while citrate alone would probably not be a good predictor of sepsis, in an ensemble of biomarkers it is very predictive.

Table 3, rows 16-17 shows the results of discovering proteomic relational biomarkers for early- and late-onset preeclampsia given public data (Chen, et al., Maternal plasma proteome profiling of biomarkers and pathogenic mechanisms of early-onset and late-onset preeclampsia, Sci Rep (2022) 12:19099). As with the previous examples, our Discovery algorithm found the proteins that the original authors called out as especially predictive. For example, the authors identify inter-alpha-trypsin inhibitor heavy chain H2-4 and our Discovery algorithm pairs it with 13 other proteins in its best-ranked ensemble of relational biomarker. But the Discovery algorithm also finds other proteins in the authors' own data that they did not identify as significant biomarkers. One example is gelsolin, which is probably involved in preeclampsia (e.g., Tannetta, et al., Investigation of the actin scavenging system in pre-eclampsia, Eur J Obstet Gynecol Reprod Biol (2014) 172:32-35). In our analysis, gelsolin paired with complement factor B gave a sensitivity and specificity of 0.82 and 1.0, respectively, for predicting early onset preeclampsia using the authors' own data.

Two analyses of canine lymphoma data demonstrate that the methods herein are not limited to human subjects. Canine lymphoma is a common blood cancer in dogs. In one study of seven dogs with canine lymphoma and seven dogs without the disease, the methods herein discovered gene expression relational biomarkers that classified the subject animals with perfect accuracy (Table 3, row 18). In a separate study of 25 dogs undergoing chemotherapy for canine lymphoma, the method herein found relational biomarkers that predict with perfect accuracy which dogs would relapse early and which would relapse later (Table 3, row 19).

Like the canine lymphoma studies, our analysis of methylation patterns in spina bifida and anencephaly is based on solid tissue biopsy (Table 3, rows 20, 21). Whereas the methylation-based analysis in (Table 3, rows 1-6) works on patterns of methylation on cfDNA fragments, the neural tube defect analysis is based on methylation levels in pairs of CpG sites. For spina bifida the sensitivity and specificity are 0.95 and 0.94, respectively. For anencephaly, the sensitivity is 1.0 and specificity is 0.94.

All of the preceding examples are atemporal, meaning that an individual in the case or noncase group contributes exactly one sample and all the individuals in a case or noncase group contribute samples at roughly the same time; for example, for gestational diseases and aneuploidies, samples are typically collected at the end of the first trimester. However, the Discovery algorithm can also be run on paired samples collected from individuals at different times; for example, samples collected from each individual in both the first and second trimester of pregnancy. Two such examples are shown in rows 22 and 23 of Table 3.

In the first case, we know the samples come from preeclampsia pregnancies, but we want to discover pairs of genes that are expressed differently toward the ends of each of the first and second trimesters. The classification problem in this case is to determine whether a sample was collected early or later in pregnancy given relational biomarkers for expression levels of two genes. The Discovery algorithm found many pairs of genes that were expressed differently early and later in pregnancy, the best of which was moderately sensitive to when the samples were collected (sensitivity 0.69, specificity 0.82, row 22, Table 3).

The second example of time-based classification asks, “Can we find a single gene whose change in expression levels between the first and second trimester is diagnostic for preeclampsia?” Using data from Moufarrej, et al., (Early prediction of preeclampsia in pregnancy with cell-free RNA, Nature (2022) 602:689-694), the Discovery algorithm found many relational biomarkers—in this case single genes expressed at different times—that yielded approximately equal accuracy, the best of which had sensitivity 0.81 and specificity 0.78 (row 23, Table 3). This is a comparable accuracy to sensitivities in the range 0.71-0.88 reported by Moufarrej, et al. However, these authors used panels of 24-32 genes, whereas we have demonstrated that a relational biomarker that captures the dynamics of expression of a single gene can perform roughly as well as a whole panel of conventional biomarkers.

In sum, the methods herein achieve high sensitivity and specificity for multiple regions (chromosome, cfDNA fragments, gene promoter regions, etc.) and -omes (methylome, transcriptome, proteome) and species (human, dog). These results were obtained for multiple classes of disease/disorder (fetal aneuploidy, sex chromosome anomalies, gestational and neonatal disease, neurodegenerative disease, neural tube defects, cancer). In addition, the methods herein produce highly accurate results for samples obtained from blood plasma (liquid biopsy) and from solid tissue biopsy. The results show that a single condition such as preeclampsia can be assayed with data from multiple-omes and, intriguingly, by comparing assays taken at different times.

### Computer Implementation of the Disclosed Models

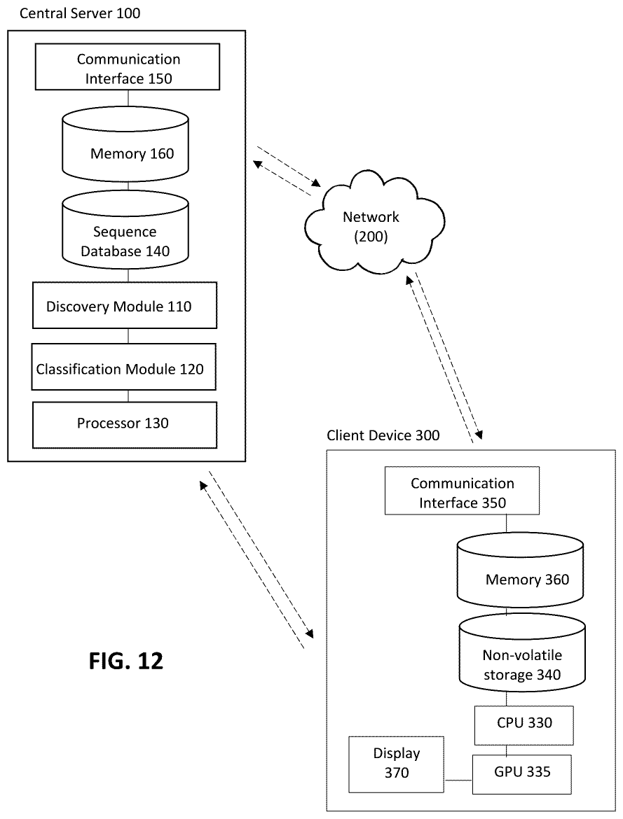

Certain of the described methods and/or their equivalents may be implemented with computer executable instructions. Thus, according to aspects of the present disclosure, a non-transitory computer readable/storage medium may be configured with stored computer executable instructions of an algorithm/executable application that, when executed by a machine(s), cause the machine(s) (and/or associated components) to perform any one of the disclosed methods. Example machines include but are not limited to a processor, a computer, a server operating in a cloud computing system, a server configured in a Software as a Service (SaaS) architecture, a smart phone, and so on. According to certain aspects, a computing device is implemented with one or more executable algorithms that are configured to perform any of the disclosed methods. For example, and with reference to FIG. 12, a system of the present disclosure may include a central server 100 that is cloud based and communicates with client devices 300 via network connections (dotted lines).

Certain of the described methods and/or their equivalents may be implemented as a system that is programmed or otherwise configured to perform the methods. As various examples, a system can process and/or assay a sample, perform sequencing analysis, measure sets of values representative of classes of molecules, identify sets of features and feature vectors from assay data, process feature vectors using a machine learning algorithm to obtain output classifications, and train a machine learning model (e.g., iteratively search for optimal values of parameters of the machine learning model).

The system can include a central processing unit 130 (CPU, also “processor” and “computer processor” herein), which can be a single core or multi core processor, or a plurality of processors for parallel processing, a memory 160 (e.g., cache, random-access memory, read-only memory, flash memory, or other memory), an electronic storage unit 140 (e.g., hard disk), communication interface 150 (e.g., network adapter) for communicating with one or more other systems, and peripheral devices, such as adapters for cache, other memory, data storage and/or electronic display. The memory, storage unit, interface, and peripheral devices may be in communication with the CPU through a communication bus (solid lines), such as a motherboard. The storage unit 140 can be a data storage unit (or data repository) for storing data.

The system of the present disclosure may be a classical computing system or may rely on quantum interference or quantum superposition to perform a computation, i.e., a quantum computing device. Quantum computing refers to a computational device or method that utilizes properties of quantum states defined by quantum mechanics such as superposition, entanglement, etc., to perform computations. Quantum devices utilize qubits which are the quantum equivalent to bits in a classical computing system. Qubits include at least two quantum states or probable outcomes. These outcomes, combined with a coefficient representing the probability of each outcome, describe the possible states, or bits of data, which can be represented by the qubits according to the principle of quantum superposition. These states can be manipulated which can shift the probability of each outcome or additionally add additional possible outcomes to perform a calculation, the final state of which can be measured to achieve a result. Thus, a major advantage of quantum computing over classical solvers and computer systems is observed in terms of scaling and sampling. While classical solvers rely on sampling one state at a time, quantum annealing can consider multiple states of a search space, and therefore, may determine multiple possible solutions simultaneously.

The system can be operatively coupled to a computer network 200 (“network”) with the aid of the communication interface 150. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing over the network (“the cloud”) to perform various aspects of analysis, calculation, and generation of the present disclosure, such as, for example, using a machine learning classifier to obtain output classifications and/or training a machine learning model. Such cloud computing may be provided by cloud computing platforms such as, for example, Amazon Web Services (AWS), Microsoft Azure, Google Cloud Platform, and IBM cloud. The network, in some cases with the aid of the computer system, can implement a peer-to-peer network, which may enable devices coupled to the computer system to behave as a client or as a server.

The processor can execute a sequence of machine-readable instructions, which can be embodied in a program or software, e.g., the discovery module 110 and classification module 120. The instructions can be stored in memory 160. The instructions can be directed to the processor 130, which can subsequently program or otherwise configure the processor to implement the methods of the present disclosure.

The storage unit 140 can store files, such as drivers, libraries and saved programs. The storage unit can store user data, e.g., user preferences and user programs. The storage unit 140 can store sequence data collections from various populations. The storage unit 140 may be part of the central server 100 or may be a remote database. The computer system in some cases can include one or more additional data storage units that are external to the computer system, such as located on a remote server that is in communication with the computer system through an intranet or the Internet.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processor adapted to execute the code, such as on a processor 330 of a client device 300 or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology can be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a host computer into the computer platform of an application server. Thus, another type of media that can bear the software elements includes optical, electrical, and electromagnetic waves, such as used across physical interfaces between local devices, through wired and optical landline networks and over various air-links. The physical elements that carry such waves, such as wired or wireless links, optical links, or the like, also can be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium, or physical transmission medium. Non-volatile storage media include, for example, optical or magnetic disks, such as any of the storage devices in any computer(s) or the like, such as can be used to implement the databases, etc. shown in the drawings. A “database,” as used herein, may refer to a digitally stored data in the form of a table, a set of digitally stored tables, and a set of data stores (e.g., disks) and/or methods for accessing and/or manipulating those data stores. Exemplary databases may comprise multiomic data and variables that may be used to distinguish a sample as a disease or disorder sample or a noncase sample and/or multiomic data that may be used to generate relational biomarkers according to the methods disclosed herein. Volatile storage media include dynamic memory, such as main memory of such a computer platform. Tangible transmission media include coaxial cables, copper wire and fiber optics, including the wires that comprise a bus within a computer system.

Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave. or any other medium from which a computer may read programming code and/or data. Many of these forms of computer readable media can be involved in carrying one or more sequences of one or more instructions to a processor for execution.

The computer system can include or be in communication with an electronic display that comprises a user interface (UI) for providing, for example, a current stage of processing or an output of the processing. Inputs are received by the computer system from one or more measurement devices. Examples of UIs include, without limitation, a graphical user interface (GUI) and web-based user interface.

Unless explicitly stated or otherwise clear from the context, the verbs “execute” and “process” are used interchangeably to indicate execute, process, interpret, compile, assemble, link, load, any and all combinations of the foregoing, or the like. Therefore, embodiments that execute or process computer program instructions, computer-executable code, or the like can suitably act upon the instructions or code in any and all of the ways just described.

The computer readable program instructions may execute entirely on a client device 300, partly on the client device, as a stand-alone software package, partly on the client computer and partly on a remote computer or entirely on the remote computer or server. In the latter scenario, the remote computer (e.g., 100) may be connected to the client computer 300 through any type of network, including a local area network (LAN) or a wide area network (WAN), or the connection may be made to an external computer (for example, through the Internet using an Internet Service Provider).

A “client device,” 300 as used herein, may be a computing device typically having a display screen 370 with a user input (e.g., touch, voice, keyboard), a memory 360, non-volatile storage 340, a communication interface 350 to send/receive communications via a network (e.g., 200) and a processor (CPU 330 and GPU 335) for computing. Client devices can include handheld devices, mobile phones, smart phones, laptops, tablets, e-readers, virtual and/or augmented reality devices, desktop computers, mainframe computers, and the like. Client devices may communication via internet, intranet, extranet, and the like.

In various implementations, a set of APIs, such as RESTful APIs, may be available for a client to integrate the systems and software disclosed herein with their existing application(s), or to allow for customization and optimization. The APIs may provide similar functions as available via the cloud platform. In some designs, the APIs may be available for both desktop and mobile applications. Some exemplary APIs may also be designed for the companies interested in utilizing the systems disclosed herein for clinical studies, such as for monitoring and data gathering purposes.

According to certain examples, a mobile application or “mobile app” may be designed to provide any of the methods disclosed herein. The mobile app may run on the client device and allow access to databases, such as described hereinabove. In some examples, the mobile app may allow a user to distinguish a sample as a disease or disorder sample or a noncase sample and/or may provide access to multiomic data that may be used to generate relational biomarker according to the methods disclosed herein.

According to certain examples, cloud platforms may include a cloud-based central server configured to act as a hub or the platform for the entire solution, and facilitate database access, client account configuration, and communication among multiple clients and client interfaces. Exemplary cloud-based central servers include at least Amazon web services (AWS), Microsoft Azure, IBM cloud, and Google cloud.

### Machine Learning Methods for Disease Classification and Prediction

The methods of the present disclosure are novel forms of supervised, discriminative machine learning (ML). As with other ML applications, the first step is to learn models and parameters given training data and the second is to classify new samples using the learned models and parameters. The disclosed Discovery method learns relational biomarkers, which as disclosed are mathematical models F(X, θ+) and F(X, θ⁻) for cases and noncases, respectively. The disclosed Classification algorithm uses these models to classify new samples in three inference regimes: case-noncase (having a disease or not), case-case (having one of two diseases) and multiclass (zero or more disease). Accordingly, the disclosed methods generally include receiving, by one or more processors, data related to a plurality of biological materials derived from a subject or plurality of subjects, and identifying, by the one or more processors, a training subset of the plurality of biological materials.

For example, when the biological material is nucleic acid, the disclosed method includes receiving, by one or more processors, a plurality of sequence reads of the nucleic acid (e.g., cfDNA, cfDNA and cffDNA mixture, gDNA), wherein the sequence reads may be from a database or may be acquired from a sequencing device. When the biological material is protein, the disclosed method includes receiving, by one or more processors, a plurality of sequence reads of the protein, fragmentation sizes of the protein, or concentration amounts of the protein, wherein the sequence reads may be from a database or may be acquired from a sequencing device, mass spectrometer, or chromatography device.

The methods may include using the training subset of the plurality of biological materials to discover relational biomarkers based on characteristics of the biological material, e.g., frequency of a genomic pattern such as methylation on nucleic acids. These relational biomarkers may be used to stratify individuals or detect disease or disorder as described herein. For example, a relational biomarker for a noncase state may be determined by defining how a pattern of a first biological material is related to a pattern of a second, third, and/or additional biological material, such as methylation patterns of regions of cfDNA, of noncase samples: F(Y, X₁, X₂, . . . , θ⁻) (i.e., samples absent the disease or disorder of interest). As discussed, Y may be the frequency of a pattern of the first biological material, while X₁, X₂, . . . , represent the frequencies of the patterns on the second, third, etc., biological materials. A function that defines the relationship between Y and X₁, X₂, . . . , is F and this function has parameters θ. Thus, the methods find a relational biomarker F(R, θ⁻) for the noncase state, where R is a matrix of frequencies of the patterns in specific biological materials, that is, R=(Y, X₁, X₂, . . . ). A relational biomarker for a case state F(R, θ+) may be determined by defining how the frequency of the pattern of the first biological material is related to the frequencies of the patterns on the second, third, and/or additional biological materials of case samples, i.e., F(Y, X₁, X₂, . . . , θ⁺) (i.e., samples having the disease or disorder of interest).

Of note, the patterns Y and X may be of the same type, e.g., hypermethylation, or different, e.g., expression levels of different genes. Moreover, the functions, i.e., F, can be linear or non-linear, and the disclosed methods do not constrain the form of F.

The above indicated method may be repeated using different subsets of the plurality of biological materials as input (i.e., subsampling). In this case, the proportion of subsamples that are correctly classified by a pair of relational biomarkers can serve as a level of confidence in the relational biomarkers.

In certain preferred aspects, the methods may define a relationship between the frequency of a pattern of just two biological materials, thus providing for lower-order (i.e., sparse) relational biomarkers that reduce the probability of overfitting, i.e., finding F(R, θ⁻) and F(R, θ+) wherein R=(Y, X). Various combinations of first and second biological materials may be compared to find the combination that offers the greatest difference between case and noncase. The relational biomarkers may be ranked based on their accuracies at discriminating cases from noncases (e.g., MCC scores, defined above).

The disclosed methods may evaluate an unknown sample to classify it as case or noncase. For example, the methods may calculate a first squared deviation of a predicted frequency of the pattern from the sample frequency of the pattern calculated using the relational biomarker for the noncase state (δ⁺=(Y−ctrl_Y_predicted)²), and a second squared deviation of the predicted frequency of the pattern from the sample frequency of the pattern calculated using the relational biomarker for the case state (δ⁺=(Y−case_Y_predicted)²). The sample may then be classified as noncase or case based on comparing the first and second squared deviations. When the first squared deviation is less than the second squared deviation, the biological sample of the subject may be classified as noncase. However, should the first squared deviation be greater than the second squared deviation, the biological sample of the subject may be classified as case.

The method may further include establishing a second, third, etc., relational biomarker for each group of case and noncase samples, i.e., an ensemble of relational biomarkers, which define multiple relationships between frequencies of a pattern of the additional biological materials of the case and noncase samples, respectively. That is, the method may find F(Rᵢ, θ⁻) and F(Rᵢ, θ+), wherein i=1, . . . , n represents n unique combinations of biological materials. For example, when the biological material is DNA and the pattern is a methylation pattern, four different methylation patterns on pairs of chromosomes would give rise to (4*23)*(4*22)/2=4048 possible pairs of relational biomarkers (one each for the case and noncase states). As another example, 1000 genes give rise to 499500 relational biomarkers in which X and Y are each a gene expression level. Pairs of relational biomarkers may be ranked based on their accuracies at discriminating cases from noncases (e.g., MCC scores, defined above).

Subsets of these pairs of relational biomarkers may be selected to serve as ensembles of relational biomarkers (Table 2). Obviously, the number of subsets of an already large number of relational biomarker pairs may be intractably large, but the accuracies of these pairs may be used to narrow the search for ensembles. Furthermore, the purpose of an ensemble of pairs of relational biomarkers is to ensure that samples that are misclassified by one pair of relational biomarkers will be correctly classified by others, so classification errors may be used to direct the search for ensembles.

Binary classifiers (i.e., case-noncase or case-case classifiers) may be selected to serve in ensembles of classifiers (FIG. 10). This is particularly valuable for multiclass inference that discriminates between several health states.

As indicated above, the methods of the present disclosure may be executed on a classical computing system or a quantum device. Thus, the present disclosure may include a quantum computer enhanced machine learning algorithm enabled via a quantum computer. Such an implementation recognizes that a quantum decision making system, such as a quantum classifier, a quantum regressor, a quantum controller, or a quantum predictor, may be used to analyze input data and make a decision regarding the input data by a quantum classifier. For example, a quantum classifier, such as a quantum support vector machine (QSVM), may be used to analyze input data and determine a discrete classification of the input data by a quantum processor. A quantum classifier, such as a QSVM, implements a classifier using a quantum processor which has the capability to increase the speed of classification of certain input data.

### ASPECTS OF THE DISCLOSURE

The present disclosure provides, according to a first aspect, a method to discover relational biomarkers that distinguish a disease or disorder case sample from a noncase sample (e.g., Table 3, rows 1-6), or distinguish between two diseases or disorders (e.g., Table 3, rows 12, 15). Additionally, the method may distinguish between more than two diseases or disorders (see FIG. 10 and discussion). The method may comprise defining a case relational biomarker by determining a relationship between a pattern of a first biological material and a pattern of at least one additional biological material of biological samples from a plurality of subjects who have or will develop the disease or disorder, and defining a noncase relational biomarker by determining a relationship between the pattern of the first biological material and the pattern of the at least one additional biological material of biological samples from a plurality of subjects who do not have and will not develop the disease or disorder. The case and noncase relational biomarkers may be used in a classification algorithm to distinguish an unknown sample as a case sample or a noncase sample, i.e., as a sample from a subject who has or will develop the disease or disorder or from a subject who does not have and will not develop the disease or disorder.

According to the aforementioned aspect, the method may further comprise generating an ensemble of case and noncase relational biomarkers by repeating the determining step for additional combinations of biological materials for each of the plurality of subjects who have or will develop the disease or disorder and each of the plurality of who do not have and will not develop the disease or disorder. The method may also comprise generating ensembles of case-noncase classifiers, such as by finding case and noncase relational biomarkers that demonstrate the greatest difference between case-noncase contrasts. The method may further generate a discriminator that comprises a case-noncase relational biomarker pair for the disease or disorder, wherein the method further comprises generating an ensemble of discriminators for additional diseases or disorders by repeating the determining step for combinations of biological material from subjects who have or will develop each additional disease or disorder, and from subjects who do not have and will not develop each additional disease or disorder.

The present disclosure further provides, according to a second aspect, a method to model system behavior. The method may comprise generating a discriminator that classifies a biological sample as from a subject who has or will develop a disease or disorder, i.e., case sample, or from a subject who does not have and will not develop a disease or disorder, i.e., noncase sample, wherein the discriminator comprises a relational biomarker pair. The relational biomarker pair may be determined by modeling a relationship between patterns of a first biological material and at least one additional biological material for each of the plurality of case samples to define a case relational biomarker, and for each of the plurality of noncase samples to define a noncase relational biomarker.

According to the aforementioned aspect, the method may further comprise generating an ensemble of discriminators by repeating the modeling step for additional combinations of biological material for each of the case and noncase samples, wherein one or more discriminators of the ensemble of discriminators may be used to distinguish the biological sample as a case sample, i.e., sample from a subject who has or will develop the disease, or a noncase sample, i.e., sample from a subject who does not have and will not develop the disease or disorder or from a subject who has or will develop a different disease or disorder. The method may further comprise generating an ensemble of discriminators for additional diseases or disorders by repeating the modeling step for combinations of biological material from subjects who have or will develop each additional disease or disorder, and from subjects who do not have and will not develop each additional disease or disorder, wherein the ensemble of discriminators may be used to distinguish the biological sample as a sample from (i) from one of the additional diseases, (ii) a noncase sample, or (iii) a no-call sample. The method may further yet comprise generating ensembles of case-noncase classifiers, such as by finding case and noncase relational biomarkers that demonstrate the greatest difference between case-noncase contrasts.

The present disclosure further provides, according to a third aspect, a method to distinguish a biological sample as a case sample, i.e., sample from a subject who has or will develop the disease, or a noncase sample, i.e., sample from a subject who does not have and will not develop the disease or disorder. The method may comprise defining a set of relational biomarkers that classify the biological sample as a case sample or a noncase sample, wherein the set of relational biomarkers include: a case relational biomarker defined by case parameters that model a relationship between patterns of at least two biological materials for each of a plurality of case samples, and a noncase relational biomarker defined by noncase parameters that model a relationship between the patterns of the at least two biological materials for each of a plurality of noncase samples. The method may use each of the case relational biomarker and the noncase relational biomarker to calculate a predicted case value and a predicted noncase value, respectively, of the pattern of the biological sample for a first of the at least two biological materials based on an empirical value of another of the at least two biological materials. The biological sample may then be classified as a case sample or a noncase sample by comparing the predicted case value and the predicted noncase value to an empirical value of the first of the at least two biological materials.

According to the aforementioned aspect, the step of comparing the expected case value and the expected noncase value to the empirical value of the first of the at least two biological materials may comprise calculating a first squared deviation of the predicted case value of the pattern from the empirical value of the pattern, and a second squared deviation of the predicted noncase value of the pattern from the empirical value of the pattern, wherein when the first squared deviation<the second squared deviation, the biological sample is classified as a case sample, and when the first squared deviation>the second squared deviation, the biological sample is classified as a noncase sample. Additionally, a subthreshold difference between these squared deviations may be used to classify samples as “no-call”.

Additionally, or alternatively, the step of comparing the expected case value and the expected noncase value to the empirical value of the first of the at least two biological materials may comprise resampling data from each of the plurality of case samples to generate case pseudosamples, and each of the plurality of noncase samples to generate noncase pseudosamples. The method further includes calculating a first squared deviation of the predicted case value of the pattern from the empirical value of the pattern for each of the case pseudosamples, and a second squared deviation of the predicted control value of the pattern from the empirical value of the pattern for each of the noncase pseudosamples. The method may classify the biological sample as a case sample if at least a threshold proportion of pseudosamples are so classified, and conversely classifying the biological sample as a noncase sample if less than a threshold proportion of pseudosamples are so classified. Additionally, subthreshold proportions may be used to classify samples as “no-call”. For example, the biological sample may be classified as: (i) case if a proportion of pseudosamples greater than (1-T), where T is a threshold, is so classified, or (ii) control if less than a proportion T of pseudosamples is so classified, or (iii) no-call if the proportion lies between T and (1-T).

According to the aforementioned aspect, the method may include generating an ensemble of the case and noncase relational biomarkers by repeating the defining step for additional combinations of biological material from each of the plurality of case and noncase samples, respectively. The method may generate a discriminator that comprises a case-noncase relational biomarker pair for the disease or disorder, wherein the method further comprises generating an ensemble of discriminators for additional diseases or disorders by repeating the determining step for combinations of biological material from subjects who have or will develop each additional disease or disorder, and from subjects who do not have and will not develop each additional disease or disorder.

According to any of the aforementioned aspects, the pattern may comprise a mathematical transformation of a frequency of a state of the biological material.

According to any of the aforementioned aspects, the biological sample may comprise one or more biofluid or samples of the subject.

According to any of the aforementioned aspects, the biological material may be nucleic acid(s), protein(s) (e.g., antibodies, enzymes, antigens, etc.), lipids, metabolite(s), or any other measurable material derived from a biological sample.

According to any of the aforementioned aspects, when the biological material is a nucleic acid, the state of the nucleic acid may comprise a methylation pattern, gene expression pattern, DNA fragmentome pattern, RNA expression pattern, RNA isoform, estimated fetal fraction of hypermethylated cfNA, or estimated fetal fraction of hypomethylated cfNA.

According to any of the aforementioned aspects, when the biological material is a nucleic acid, the first region and the at least one additional region of the nucleic acid comprises: the same nucleic acid region from biological samples taken at different time points; regions of the same chromosome; regions of different chromosomes; a first and at least a second gene; a first and at least a second regulatory region; a first and at least a second transcribed gene; a first and at least a second region of a transcriptome; or any combination thereof.

According to any of the aforementioned aspects, when the biological material is a protein, the state of the protein may comprise abundance or localization of one or more proteins, proteoforms, posttranslational modification(s), degradation state (fragment sizes, distribution, abundance, etc.), or protein interaction partner(s).

According to any of the aforementioned aspects, when the biological material is a protein, the first region and the at least one additional region of the protein comprises: different proteins; the same protein from biological samples taken at different time points; the same protein from different locations in a cell, organ, or organism; or any combination thereof.

According to any of the aforementioned aspects, when the biological material is a metabolite, the first region and the at least one additional region of the metabolite comprises: different metabolites; the same metabolite taken from different biofluid or tissue samples, such as from different locations in a cell, organ, or organism; the same metabolite from biological samples taken at different time points; or any combination thereof.

According to any of the aforementioned aspects, the first region and the at least one additional region of the biological material may be selected to maximize an estimated classification accuracy of the case and noncase relational biomarkers.

The present disclosure provides one or more computer storage media having computer-executable instructions embodied thereon that, when executed, perform any of the disclosed methods, such as any of the disclosed methods according to the first, second, and/or third aspects.

The present disclosure further yet provides systems for distinguishing disease or disorder samples, i.e., case samples, from noncase samples. The system generally comprises a server coupled to a wireless network and configured to communicate with a plurality of client devices. The server comprises a processor configured to execute instructions that perform any of the methods disclosed herein, and may be coupled to a database comprising multiomic data and/or variables that may be used by the processor, such as to (i) distinguish a sample as a disease or disorder sample or a noncase sample, (ii) discover relational biomarkers that discriminate disease or disorder samples from noncase samples, and/or (iii) model system behavior that distinguishes disease or disorder samples from a noncase samples. The computer-readable instructions may be stored on a memory of the server. Alternatively, the system may be configured to execute all of or only a portion of the instructions that perform any of the methods disclosed herein on a processor of a client device.

### General Abbreviations and Definitions

Definitions of certain terms used throughout this application are presented in Table 1. Additional terms used in this application are detailed herein below.

Throughout this description and in the appended claims, use of the singular includes the plural and plural encompasses singular, unless specifically stated otherwise. For example, although reference is made herein to “a” sample or “the” region, one or more of any of these components and/or any other components described herein can be used. As another example, the term “a nucleic acid” includes a plurality of nucleic acids, including mixtures thereof.

The term “comprises,” and grammatical equivalents thereof are used herein to mean that other components, ingredients, and steps, among others, are optionally present. For example, an embodiment “comprising” (or “which comprises”) components A, B, and C can consist of (i.e., contain only) components A, B, and C, or can contain not only components A, B, and C but also contain one or more other components. In the present disclosure, all embodiments where “comprising” is used may have as alternatives “consisting essentially of,” or “consisting of.” In the present disclosure, any method or apparatus embodiment may be devoid of one or more process steps or components. In the present disclosure, embodiments employing negative limitations are expressly disclosed and considered a part of this disclosure.

Furthermore, use of “or” means “and/or” unless specifically stated otherwise. “Including” and like terms means including, but not limited to. When ranges are given, any endpoints of those ranges and/or numbers within those ranges can be combined within the scope of the present invention.

Other than in any operating examples, or where otherwise indicated, all numbers expressing, for example, confidence levels used in the specification and claims are to be understood as being modified in all instances by the term “about.” Accordingly, unless indicated to the contrary, the numerical parameters set forth in the preceding specification and appended claims are approximations. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

In the present disclosure, various features may be described as being optional, for example, through the use of the verb “may;” or, through the use of any of the phrases: “in some embodiments,” “in some implementations,” “in some designs,” “in various embodiments,” “in various implementations,” “in various designs,” “in an illustrative example,” or “for example,” or through the use of parentheses. For the sake of brevity and legibility, the present disclosure does not explicitly recite every permutation that may be obtained by choosing from the set of optional features. However, the present disclosure is to be interpreted as explicitly disclosing all such permutations. For example, a system described as having three optional features may be embodied in seven separate ways, namely with just one of the three possible features, with any two of the three possible features or with all three of the three possible features.

In the present disclosure, the term “any” may be understood as designating any number of the respective elements, that is, as designating one, at least one, at least two, each or all of the respective elements. Similarly, the term “any” may be understood as designating any collection(s) of the respective elements, that is, as designating one or more collections of the respective elements, a collection comprising one, at least one, at least two, each or all of the respective elements. The respective collections need not comprise the same number of elements.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

As used herein, the term “subject,” generally refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. A subject can be an invertebrate, such as, for example, an arthropod (e.g., insects and crustaceans) or nematode. The subject can be absent a disease or disorder. Alternatively, the subject may have a disease or disorder of interest or can be absent observable symptoms of the disease or disorder of interest. That is, the subject may be displaying a symptom(s) indicative of a health or physiological state or condition of the subject, such as a cancer or other disease, disorder, or condition of the subject, or can be asymptomatic with respect to such health or physiological state or condition.

As used herein, the term “biological sample,” “patient sample,” or “sample” refers to any sample taken from a subject, which can reflect a biological state associated with the subject, and that includes nucleic acids or fragments thereof, proteins or fragments thereof, metabolites or fragments thereof, lipids or fragments thereof, and the like. A biological sample can be a bodily fluid (“biofluid”) such as blood, plasma, serum, urine, vaginal fluid, fluid from a hydrocele (e.g., of the testis), vaginal flushing fluids, menstrual fluid, cervical fluid, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological material can be a stool sample. A biological material can be a tissue sample such as a biopsy from an organ or any tissue. A biological sample can be an intracellular or extracellular extract or preparation. A biological sample can be a preparation of a subcellular compartment, i.e., organelle, vesicle, etc., from the intracellular or extracellular space.

Biological samples may be cell-free biological samples or substantially cell-free biological samples or may be processed or fractionated to produce cell-free biological samples. A “cell-free” nucleic acid can be greater than 50%, 60%, 70%, 80%, 90%, 95%, or 99% cell-free. A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis. Cell-free biological samples may be obtained or derived from subjects using an ethylenediaminetetraacetic acid (EDTA) collection tube, a cell-free RNA collection tube, a cell-free DNA collection tube, or any other method known in the art. Cell-free biological samples may be derived from whole blood samples by fractionation (e.g., centrifugation into a cellular component and a cell-free component). Biological samples or derivatives thereof may contain cells. For example, a biological sample may be a blood sample or a derivative thereof (e.g., blood collected by a collection tube or blood drops).

The sample may be a fresh or archival sample derived from a subject having a disease or disorder, such as a cancer selected from the group consisting of prostate cancer, metastatic prostate cancer, ovarian cancer, breast cancer, triple negative breast cancer, lung cancer, multiple myeloma, pancreatic cancer, and colon cancer. While exemplary cancers are listed, other cancers are possible and within the scope of the methods of the present disclosure.

The sample may be a fresh or archival sample derived from a subject having a disease or disorder other than cancer, such as diabetes, kidney disease, Alzheimer's disease, myocardial infarction, stroke, autoimmune disorders, transplant rejection, multiple sclerosis, type I diabetes, preterm birth, preeclampsia, endometriosis, necrotizing enterocolitis, a brain condition, or any disease that results in an increase in cell death. For example, an increase in apoptotic or necrotic cell death. Here again, while exemplary diseases are noted, others are possible and within the scope of the methods of the present disclosure.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs.

The term “cell free NA” or “cfNA” refers to nucleic acid fragments that circulate in an individual's body and encompasses nucleic acid fragments that originate from normal cells and/or tumor cells or other types of cancer cells, and which may be released into a bodily fluid of an individual (e.g., blood, sweat, urine, or saliva) as result of biological processes such as apoptosis or necrosis of dying cells or actively released by viable normal or tumor cells. The term “normal cells” should be understood to indicate only that the cells are not cancer cells, and thus could be cells of a subject having a disease or disorder of interest, or from a subject absent the disease or disorder of interest. Cell free DNA includes cell-free deoxyribonucleic acid (cfDNA), cell-free fetal DNA (cffDNA), genomic DNA (gDNA), from a liquid or tissue sample of a subject. cfDNA can comprise both nuclear cfDNA (n-cfDNA) and mitochondrial cfDNA (mt-cfDNA), as well as circulating tumor DNA (ctDNA) and donor-derived cfDNA (dd-cfDNA). The term nucleic acid may also refer to cell free RNA (cfRNA), or a combination of cfRNA and cell free fetal RNA (cffRNA), wherein cell free RNAs can comprise mRNA, rRNA, tRNA, snRNA, siRNA, snoRNA, miRNA, circRNA, lncRNA, endoRNA, YRNA, and the like.

The term “genomic nucleic acid” or “gNA” refers to nucleic acid molecules, such as deoxyribonucleic acid molecules, obtained from one or more cells. The gNA can be extracted from healthy cells (e.g., non-tumor cells) or from non-healthy cells (e.g., a biopsy sample). In some embodiments, gNA can be extracted from a cell derived from a blood cell lineage, such as a white blood cell.

The term “methylation” or “DNA methylation” refers to addition of a methyl group to a nucleotide base in a nucleic acid molecule. In some embodiments, methylation refers to addition of a methyl group to a cytosine at a CpG site, cytosine-phosphate-guanine site (i.e., a cytosine followed by a guanine in a 5→3′ direction of the nucleic acid sequence). In some embodiments, DNA methylation refers to addition of a methyl group to adenine, such as in N6-methyladenine. In some embodiments, DNA methylation is 5-methylation (modification of the 5th carbon of cytosine). In some embodiments, 5-methylation refers to addition of a methyl group to the 5C position of the cytosine to create 5-methylcytosine (5mC). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the 3rd carbon of cytosine). In some embodiments, 3C methylation comprises addition of a methyl group to the 3C position of the cytosine to generate 3-methylcytosine (3mC). Methylation can also occur at non CpG sites, for example, methylation can occur at a CpA, CpT, or CpC site.

The term “hypermethylation” refers to an increased level or degree of methylation of nucleic acid molecule(s) relative to the other nucleic acid molecules within a population (e.g., sample) of nucleic acid molecules. In some embodiments, hypermethylated DNA can include DNA molecules comprising at least 1 methylated residue, at least 2 methylated residues, at least 3 methylated residues, at least 5 methylated residues, or at least 10 methylated residues.

The term “hypomethylation” refers to a decreased level or degree of methylation of nucleic acid molecule(s) relative to the other nucleic acid molecules within a population (e.g., sample) of nucleic acid molecules. In some embodiments, hypomethylated DNA includes unmethylated DNA molecules. In some embodiments, hypomethylated DNA can include DNA molecules comprising no methylated residues or an overall increase in the unmethylated state of CpG sites in sequences that are normally methylated.

The term “machine learning model” refers to a collection of parameters and functions, where the parameters are trained on a set of training samples. The parameters and functions may be a collection of linear algebra operations, non-linear algebra operations, and tensor algebra operations. The parameters and functions may include statistical functions and probability models. The training samples generally include sequence reads, or a first or second, etc. subset of sequence reads. In supervised machine learning the training samples are augmented with known classifications/labels (e.g., case or noncase) for the samples. In unsupervised machine learning and semi-supervised machine learning, these labels are missing for all or some samples.

While multiple embodiments are disclosed, still other embodiments of the present inventions will become apparent to those skilled in the art from this detailed description. The inventions are capable of myriad modifications in various obvious aspects, all without departing from the spirit and scope of the present invention. Accordingly, the drawings and descriptions are to be regarded as illustrative in nature and not restrictive.

What is claimed is:

1. A method to discover relational biomarkers comprising:
for biological samples from subjects who have or will develop a disease or disorder (case samples), determining a case relationship between a pattern of a first biological material and a pattern of at least one additional biological material from each of a plurality of case samples; and
for biological samples from subjects who do not have or will not develop the disease or disorder (noncase samples), determining a noncase relationship between the pattern of the first biological material and the pattern of the at least one additional biological material from each of a plurality of noncase samples,
wherein the biological material comprises a methylation marker, nucleic acid, protein, or metabolite,
identifying a plurality of biomarkers, each representing the case relationship and the non case relationship, and
modeling a mathematical relationship of the plurality of biomarkers to generate at least one relational biomarker.

2. The method of claim 1, comprising:
generating an ensemble of the relational biomarkers by modeling combinations of the mathematical relationships of additional biomarkers from biological material from each of the plurality of case and noncase samples, respectively.

3. The method of claim 1, wherein a discriminator comprises at least one relational biomarker for classifying an unknown biological sample as a case or noncase sample for the disease or disorder, and the method comprises:
generating an ensemble of discriminators for additional diseases or disorders by modeling combinations of the mathematical relationships of additional biomarkers from the first and at least one additional biological material from subjects who have or will develop each additional disease or disorder, and from subjects who do not have or will not develop each additional disease or disorder.

4. The method of claim 1, wherein the biological material comprises: at least a methylation marker; individual nucleic acids, regions of nucleic acids, or groups of nucleic acids; individual proteins or groups of proteins; individual metabolites or groups of metabolites; and any combinations thereof.

5. The method of claim 1, wherein the pattern comprises a variable representing a presence or absence or quantity or frequency or proportion of the first and at least one additional biological material, or a mathematical transformation of the variable.

6. The method of claim 3, wherein the first and the at least one additional biological material are selected to maximize an expected accuracy of the discriminator for classifying the unknown biological sample as a case or noncase sample for the disease or disorder.
